SEQUENCE LISTING (10 sequences)

SEQ ID NO: 1 - DNA, 1001 bp, Homo sapiens
gaccaaattcagcctactgcctcggatctctccagccgaagtctgtggactgcaagtctttgagatgcttctggctcccatcacctctaacatccttgtctgggtctaccaggaacgcttcatttccttggggctgcagttttgtggttgaggggcctggagaaaatcattttctccccttggcagtgtcccagggccctggatggtcctcttaccaacatctggtcttccaggcactcaaaagctgggaaccagcatctcagcgccagctctaccagttctcgttttgggccagaggcagcctctgcactcccacgcctgtcctcctggaagggacctggttggactaacggctaacctggacctggaactgtagggccaggggattgtctcagggccgacgttccacctggggcttccctccccacccaccccgactccaggctttcccttttttcttttgttcaacattgtaagaacaatcaatgctgttattactgttcccaccatgattgatgtggggtaaatattaaggagatggcctcatgggaatttgaccttgactagaaatagagactgagagtgagcaaccagctggaaggtactatgccagtcctagcagaaaaatgtgttaggggcctggcccaaagcagtgttggttgcttacagtgttgattgattttgttcttttttcttaccacctcttttctttccctctcatggtacctgctcatggttatgaagctttcaaagtaaagaacacgaaatacctcccaagtattaccagtgggtaccaaaaaaatgtccccttgagtcttttccttgtttttagatgttaattctctcccttggcatccggttagccccccagggggggcagcattgtggagaacttgatatttagttactgatgctcttccaggacacgaaaagaacccatctttgaatatcaatgattttttttttttaagtactgttccggggagaaaaacagtctcaaa

SEQ ID NO: 2 - DNA, 1001 bp, Homo sapiens
attagcaaccttacatctactactttagtttctttttgccatgtaacataacacattcacaggatccagggattaggacacagatgtcttgtgggagagggaacattattctgcctaccacatgcatacatcagaaaccatggttgaaacacaggaaacatgacagttcctcaaggcatacaattatgaccttgttgggttaaccttcactatccaaattttaatcacacaaacttttccttaatctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatcgtgagggcagcaaaggaggccaacatacatgccttcatcgagtcactgcctaatgtaagtctctcttcaaataaacagcctgggagcatgtggcagcctctctggcctatagtttgatttataaggggctggtctcccagaagtgaagagaaattagcaaccaaatcacacccttacctgtatacaagcatctggccacacttcctgtttgggttagttgttacctttacctgatcacctgaccctccttgtgaggaagggatgaaagtgttcgaccacttcaggtttaggagagaggaacatttctgggataggagaactggaacaattgtcttgatccaaagctataggcttgaggctccacctttgtcagccttaggggtaagtacaatatctggaaagcctttcactttaagtccaagtacagagtctgggtccccacctgcacatgctgcttctggcctgctgaggaagtaggcatgactgtctctccccatgtc

SEQ ID NO: 3 - DNA, 663 bp, Homo sapiens
ctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggagcctcggaagacaggatttgcatagatgggtttgggaaagacattccagagaccccactgtaagagggcctggagaggagg

SEQ ID NO: 4 - DNA, 1001 bp, Homo sapiens
ttagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtcctgaggatgccccaccaccagcaaacatgggtggtgggtgaaaccacaggctggaccagaagccaggctgagaaggggaagcaggtttgggggacttcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgc

SEQ ID NO: 5 - DNA, 1001 bp, Homo sapiens
caaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcccctggtgatagccccagcatggctactgccaggtgggcccactctaggaaccctggccacctagtcctcaatgccaccacactgactgtccccacttgggtggg

SEQ ID NO: 6 - DNA, 1001 bp, Homo sapiens
ctcccacttctaaaatactaattcaatttcagaggctgcttgatagaaatcaatatagcagggactatctttgtagtatcaatcaggttgtgcaaactcttttaacctatgctatcatctccaaaatgttaatgtagtaattcataccatcttatatttcaagattgtagagaagaattgttgtaaaaagtaagagaattaatataaagatgcttttatactatcaaaagcaggtataagtctaggaaatgattatcatctttgattctcttgtcagaattttctttctcaaatcttgtataatcagagaattactacacatgtacaataaaaatttccccatcaagatatacaatatattttatttatatttatagttttaaattacaaccagagcttggcatattgtatctatacctttattaaatgcttttaatttaataaattattgttttctcttagatatgcaataattttcccactatcattgattatttcccgggaacccataacaaattacttaaaaaccttgcttttatggaaagtgatattttggagaaagtaaaagaacaccaagaatcgatggacatcaacaaccctcgggactttattgattgcttcctgatcaaaatggagaaggtaaaatgttaacaaaagcttagttatgtgactgcttgcgtatttgtgattcattgactagttttgtgtttactacggatgtttaacaggtcaaggagtaatgcttgagaagcatatttaagtttttattgtatgcatgaatatccagtaagcatcatagaaaatgtaaaattaaattgttaaataattagaatacatagaagaaattgtttagataaatataatctatctgaacaataaggatgtcaggataggaaaagctctgttctgcagcttccagtgagatcagcacaggaggaacttaaatttaaaagaaaataaaaaacatctccatcaaaaagtgagtgaaggatatgaacaga

SEQ ID NO: 7 - DNA, 1001 bp, Homo sapiens
ttgtctacagactttgcagactgatgtgattccctctgaaacttgaattatttggtttctaaaaaagtctctttttttctttccaaagtaaaagacaaataggccgggaatgtaaatttagcatttgagcaaccattatttaaccagctaggctgtaattgttaattcgagattaatgtaaaagtgatgtgttgattttatgcatgccaaactcttttttgcttttaagggaattcataggtaagatattacttaaaatttctaaactattattatctgttaacaaatatgaagtgttttatatctaatgtttactcatattttaaaattgtttccaatcatttagcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctggatccaggtaaggccaagttttttgcttcctgagaaaccacttacagtctttttttctgggaaatccaaaattctatattgaccaagccctgaagtacatttttgaatactacagtcttgcctagacagccatggggtgaatatctggaaaagatggcaaagttctttattttatgcacaggaaatgaatatcccaatatagatcaggcttctaagcccattagctccctgatcagtgttttttccactaaactccaaagccctgtttctataaagtactttggtgacagccccaaagcgtgcttatatcactccatggacatccaggcactttggagtcttccattactcacaaggcttgtccttcaattcacactttgtcatattgtgtgacagaaatatcctaatctaaaagacattatctccttcaaggacagagaatatttggaaccacagaagctgccaagaaacactgaatagggcagaggtgtttgatgtctc

SEQ ID NO: 8 - DNA, 1090 bp, Homo sapiens
gctcccgttaaggtctatacattgtggtggttttgtgctgtgggtccatttagtgatttccctacctcccatcctctattagattcacaactgttgttctgcccataatttcctatgcttgctttgcattgttacatttttttttgaaaatcagaaagcaaaatcaatataaagcagccatgtctggaggagaccaggaggtcaagaagccttagtttctcaagcccttagcaccaaattctctgagatcagctcttccttcagttacactgagcatttcccctctgcagtgatggagaagggagaactcttattttttctcatgagcatctctggggctgttttccttagataaataagtggttctatttaatgtgaagcctgttttatgaacaggatgaatgtggtatatattcagaataactaatgtttggaagttgttttgttttgctaaaacaaagttttagcaaacgattttttttttcaaatttgtgtcttctgttctcaaagcatctctgatgtaagagataatgcgccacgatgggcatcagaagacctcagctcaaatcccagttctgccagctatgagctgtgtggcaccaacaggtgtcctgttctcccagggtctcccttttcccatttgaaatataaaaaataacaattcctgccttcacgtgtttttttagggggttaaatggtaaaggtgtttatatctgctaaggtaatttacttgatatatgtttggttattgaagatatatgagttatgttagctatttcatgtttaggctgctgtatttttagtaggctatattaaatagaggatttcattataaaggacaaagtctcctaatcttcgatataggattgacatactttttaaatatacaaggcatagaatatggccatttccgttaaatcataaattcccaactggttattaatctaagaattcagaattttaagtaattgtttttgcatcagattgtttacttcagtgctctcaattatgacggtgcattggaaccacttgggttaacatttttttgtttttattaccaatacctaggcttcaacctagtacaatgaaa

SEQ ID NO: 9 - DNA, 151 bp, Homo sapiens
  misc_feature (76)..(76): n is a, c, g, or t
tttgtggactgacagctttttatagtcacgtgacacagtcaaacattaacttggtgtatcgattggtttttgccanagtaggagagggcgaacctctggcaggagcaaaggcgccatggctgtggagtcccagggcggacgcccacttgtc

SEQ ID NO: 10 - DNA, 1001 bp, Homo sapiens
aatactaatttaatggatcctgaggttctggaagtactttgctgtgttcactcaagaatgtgatttgagtatgaaattccagccagttcaactgttgttgcctattaagaaacctaataaagctccaccttctttatctctgaaagtgaactccctgctacctttgtggactgacagctttttatagtcacgtgacacagtcaaacattaacttggtgtatcgattggtttttgccatatatatatatataagtaggagagggcgaacctctggcaggagcaaaggcgccatggctgtggagtcccagggcggacgcccacttgtcctgggcctgctgctgtgtgtgctgggcccagtggtgtcccatgctgggaagatactgttgatcccagtggatggcagccactggctgagcatgcttggggccatccagcagctgcagcagaggggacatgaaatagttgtcctagcacctgacgcctcgttgtacatcagagacggagcattttacaccttgaagacgtaccctgtgccattccaaagggaggatgtgaaagagtcttttgttagtctcgggcataatgtttttgagaatgattctttcctgcagcgtgtgatcaaaacatacaagaaaataaaaaaggactctgctatgcttttgtctggctgttcccacttactgcacaacaaggagctcatggcctccctggcagaaagcagctttgatgtcatgctgacggaccctttccttccttgcagccccatcgtggcccagtacctgtctctgcccactgtattcttcttgcatgcactgccatgcagcctggaatttgaggctacccagtgccccaacccattctcctacgtgcccaggcctctctcctctcattcagatcacatgaccttcctgcagcgggtgaagaacatgctcattgccttttcacagaactttctgtgcgacgtggtttattccccgtatgcaacccttgcctcagaattc

(19) United States
(12) Patent Application Publication
(10) Pub. No.: US 2018/0105876 A1
(43) Pub. Date: Apr. 19, 2018

(54) MEDICATION DOSING REPORT

(71) Applicant: BAYCREST TECHNOLOGY PTY LTD, Victoria (AU)

(72) Inventor: Ajeet B. Singh, Victoria (AU)

(21) Appl. No.: 15/567,445

(22) PCT Filed: Apr. 15, 2016
(86) PCT No.: PCT/AU2016/050280

§ 371 Date: Oct. 18, 2017

Related U.S. Application Data
(60) Provisional application No. 62/149,529, filed on Apr. 18, 2015.

(51) Int. Cl. C12Q 1/6883

(52) U.S. Cl. CPC C12Q 1/6883; C12Q 2600/156; C12Q 2600/106

(57) ABSTRACT

The present invention relates methods and kits for determining a prognosis of a clinical response to a central nervous system (CNS)-active medicament in a patient suffering from major depressive disorder, cyclothymic disorder, or persistent depressive disorder.

### FIELD OF THE INVENTION

The present invention relates to methods and kits for determining a prognosis of a clinical response to a central nervous system (CNS)-active medicament in a patient suffering from major depressive disorder, cyclothymic disorder, or persistent depressive disorder, and CNS active medicament medication dosing reports.

### BACKGROUND

The World Health Organisation predicts that major depressive disorder (MDD) will become a leading cause of disability globally by 2030. Reducing the burden of disease from MDD is a public health priority, yet it appears the per capita level of disability from MDD globally is increasing.

Antidepressants have assisted treatment of more severe MDD, with demonstrated superiority over placebo. Unfortunately, 30-50% of patients do not respond (at least a halving of the depression rating scale score), to their first antidepressant trial. Remission (return of the rating scale to normative levels, e.g. 17-item Hamilton Depression Rating Scale (HDRS)≤7) is clinically a more translatable efficacy measure as those who respond but fail to remit tend to relapse. Remission not response is the pathway to recovery from MDD. Antidepressant remission rates are even lower than those for response, as low as 37.5% according to a meta-analysis of 2971 subjects.

It can take several months (even years) of clinical trial and error before an effective tolerable antidepressant is found for an individual patient. During this time, patients remain exposed to the handicapping effects of their symptoms and the risk of acting on self-harm ideations.

There is therefore a need for improved methods for determining a prognosis of a clinical response to antidepressants for individual patients.

### SUMMARY OF THE INVENTION

An aspect of the present invention is a method of determining a prognosis of a clinical response to a central nervous system (CNS)-active medicament in a patient suffering from major depressive disorder, cyclothymic disorder or persistent depressive disorder, said method comprising determining the presence or absence of an allelic variant of ABCC1; wherein the presence or absence the allelic variant indicates an improved or worsened clinical response to the CNS-active medicament.

In an embodiment of the invention, the allelic variant of ABCC1 analysed in the method as described herein is rs212090.

In an embodiment of the invention, the presence of an A;A or A;T allelic variant of rs212090 indicates an improved clinical response to the CNS-active medicament.

In an embodiment of the invention, the presence of a T;T allelic variant of rs212090 indicates a worsened clinical response to the CNS-active medicament.

In an embodiment of the invention, the method as described herein further comprises determining the presence or absence of an allelic variant of ABCB1.

In an embodiment of the invention, the allelic variant of ABCB1 analysed in the method as described herein is rs1045642.

In an embodiment of the invention, the presence of a T;T allelic variant of rs1045642 indicates an improved clinical response to the CNS-active medicament.

In an embodiment of the invention, the presence of a C;C or C;T allelic variant of rs1045642 indicates a worsened clinical response to the CNS-active medicament.

In an embodiment of the invention, the method as described herein further comprises determining the presence or absence of an allelic variant selected from the group consisting of CYP2D6, CYP2C19 and UGT1A1.

In an embodiment of the invention, the allelic variant of CYP2D6 analysed in the method as described herein is selected from the group consisting of rs3892097, rs1065852, rs28371725 and a deletion or duplication of P450 2D6.

In an embodiment of the invention, the allelic variant of CYP2C19 analysed in the method as described herein is selected from the group consisting of rs4244285, rs4986893 and rs12248560.

In an embodiment of the invention, the allelic variant of UGT1A1 analysed in the method as described herein is selected from the group consisting of rs8175347 and rs4148323.

In an embodiment of the invention, the presence of an A;G or G;G allelic variant of rs3892097 and/or the presence of a C;C allelic variant of rs1065852 and/or the presence of a G;G allelic variant of rs28371725 indicates an improved clinical response to the CNS-active medicament.

In an embodiment of the invention, the presence of a G;G allelic variant of rs4244285 and/or the presence of a G;G allelic variant of rs4986893 and/or the presence of a C;T or T;T allelic variant of rs12248560 indicates an improved clinical response to the CNS-active medicament.

In an embodiment of the invention, the presence of a TA(7) or TA(8) homozygous allelic variant of rs8175347 and/or TT allelic variants of rs8175347 indicate an improved clinical response to the CNS-active medicament.

In an embodiment of the invention, the presence of an A;A allelic variant of rs3892097 and/or the presence of a C;T or T;T allelic variant of rs1065852 and/or the presence of an A;A or A;G allelic variant of rs28371725 indicates a worsened clinical response to the CNS-active medicament.

In an embodiment of the invention, the presence of an A;A or A;G allelic variant of rs4244285 and/or the presence of an A;A or A;G allelic variant of rs4986893 and/or the presence of a C;C allelic variant of rs12248560 indicates a worsened clinical response to the CNS-active medicament.

In an embodiment of the invention, the method as described herein further comprises the step of administering to a patient having an allelic variant associated with an improved clinical response a decreased dose of the CNS-active medicament relative to the recommended dosage of the CNS-active medicament.

In an embodiment of the invention, the method as described herein further comprises the step of administering to a patient having an allelic variant associated with a worsened clinical response an increased dose of the CNS-active medicament relative to the recommended dosage of the CNS-active medicament.

In an embodiment of the invention, the CNS-active medicament administered in the method as herein described is selected from the group comprising sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline.

In an embodiment of the invention, the CNS-active medicament administered in the method as herein described is desvenlafaxine.

In an embodiment of the invention, the CNS-active medicament administered in the method as herein described is a substrate of the ABCC1 protein and/or ABCB1 protein.

In an embodiment of the invention, the presence or absence of an allelic variant is determined by a genotyping analysis comprising the use of mass-spectrometric analysis, microarray analysis, sequencing analysis, polymerase chain reaction and/or polymorphism specific primers.

In an embodiment of the invention, the improved clinical response is selected from the group consisting of a delayed, partial, sub-optimal and no clinical response to the CNS-active medicament.

In an embodiment of the invention, the improved clinical response is selected from the group consisting of increased pharmacologic response to the CNS-active medicament, improved treatment of one or more symptoms associated with major depressive disorder, cyclothymic disorder, or persistent depressive disorder, increased remission, decreased medical absence and decreased intolerance to the CNS-active medicament.

A further aspect of the invention is a kit for determining a prognosis of a clinical response to a CNS-active medicament in a patient suffering from major depressive disorder, cyclothymic disorder, or persistent depressive disorder, said kit comprising a primer or probe for determining the presence or absence of an allelic variant of ABCC1.

In an embodiment of the invention, the allelic variant of ABCC1 analysed in the kit as herein described is rs212090.

In an embodiment of the invention, the kit as herein described further comprises a primer or probe for determining the presence or absence of an allelic variant of ABCB1.

In an embodiment of the invention, the allelic variant of ABCB1 analysed in the kit as herein described is rs1045642.

In an embodiment of the invention, the kit as herein described further comprises a primer or probe for determining the presence or absence of an allelic variant of one or more of CYP2D6, CYP2C19 and UGT1A1.

In an embodiment of the invention, the allelic variant of CYP2D6 analysed in the kit as herein described is selected from the group consisting of rs3892097, rs1065852, rs28371725 and a deletion or duplication of P450 2D6; the allelic variant of CYP2C19 analysed in the kit as herein described is selected from the group consisting of rs4244285, rs4986893 and rs12248560, and/or the allelic variant of UGT1A1 analysed in the kit as herein described is rs8175347 or rs4148323.

Another aspect of the invention is a prognostic report generated according to the method of determining a prognosis of a clinical response to a central nervous system (CNS)-active medicament in a patient suffering from major depressive disorder, cyclothymic disorder or persistent depressive disorder as herein described.

### FIGURES

FIG. 1 shows concordance between actual desvenlafaxine dose and genetically guided predicted desvenlafaxine dose required for symptom remission. Points and error bars represent means and 95% confidence intervals, respectively

### DETAILED DESCRIPTION OF THE INVENTION

The present inventor has demonstrated that the presence or absence of an allelic variant of ABCC1 can be used to determine a prognosis of a clinical response, including remission, to antidepressants in patients suffering Major Depressive Disorder (MDD), cyclothymic disorder, or persistent depressive disorder. Accordingly, in one aspect the present invention provides a method of determining a prognosis of a clinical response to a central nervous system (CNS)-active medicament in a patient suffering from major depressive disorder, cyclothymic disorder, or persistent depressive disorder, said method comprising determining the presence or absence of an allelic variant of ABCC1; wherein the presence or absence the allelic variant indicates an improved or worsened clinical response to the CNS-active medicament.

As used herein the term “prognosis” includes the prediction of the likelihood of the occurrence or extent of a clinical response described herein to a central nervous system medicament, or the likelihood of the occurrence or extent of a clinical response described herein to a particular dose or dosage range of a central nervous system medicament.

As used herein the term “clinical response”, includes a response to a central nervous system medicament that can be detected and appreciated by a change in signs and symptoms caused by a disease for which the central nervous system medicament is being taken. For example, a clinical response includes an increased pharmacologic response to a CNS-active medicament, improved treatment of one or more symptoms associated with major depressive disorder, cyclothymic disorder, or persistent depressive disorder (e.g. a reduction of the severity of a symptom of the frequency of occurrence of a symptom of major depressive disorder, cyclothymic disorder, or persistent depressive disorder), increased remission, decreased medical absence and decreased intolerance to the CNS-active medicament.

Methods for assessing a remission status is known in the art. For example, remission can be evaluated according to the Hamilton Depression Rating Scale (HDRS or HAM-D; Hamilton, Br. J. Soc. Clin. Psychol. 6 (1967) 278-296). A HAM-D score of 10 or below is regarded as remission of the depressive symptoms. A score between 0 to 7 is considered normal, i.e. without depression. A score of 20 or higher indicates moderate, severe or very severe depression. The term “clinical response” as used herein also includes a reduction of the severity of symptoms by over 10, 20, 40 or 50% from the severity of symptoms at the beginning of treatment. There are other clinical scoring systems for assessing depression such as the Montgomery-Asberg Depression Rating Scale (MDRS), the Beck Depression Inventory (BDI) and Zung Self-Rating Depression Scale, the Wechsler Depression Rating Scale, the Raskin Depression Rating Scale, the Inventory of Depressive Symptomatology (IDS), the Quick Inventory of Depressive Symptomatology (QIDS) and others. It should be appreciated that the present invention may be used in conjunction with other clinical scoring systems.

In one embodiment, the clinical response is a reduction of the HDRS to less than 10, less than 9, less than 8 or less than 7.

In another embodiment, the clinical response is an at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold or at least 4-fold greater chance of remission when compared to a non-genetically guided dosing.

As used herein the term “genetically guided”, includes determining the presence or absence of one more allelic variants as described herein, to determine a prognosis of a clinical response, or to determine a dose of a CNS-active medicament to be administered to a patient.

As used herein the term “non-genetically guided” or “genetically unguided”, includes determining a prognosis of a clinical response, or to determine a dose of a CNS-active medicament to be administered to a patient, without consideration of the presence or absence of one more allelic variants as described herein in a patient.

A clinical response may also be assessed without the use of a clinical scoring system. For example, clinical response may also be measured by the rate of medication intolerance, the proportion of patients taking medical absence (also known as sick leave) and the average length of medical absence. In one embodiment, the percentage of patients taking medical absence is reduced by at least 2-fold, at least 2.2-fold, at least 2.4-fold, at least 2.6-fold, at least 2.8-fold, at least 3-fold, at least 3.2-fold, at least 3.4-fold, at least 3.6-fold, at least 3.8-fold, at least 4-fold or at least 5-fold when compared to a non-genetically guided dosing. In a further embodiment, the average length of medical absence is reduced by at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold or at least 4-fold when compared to a non-genetically guided dosing.

Medication intolerance refers to a situation where the recommended dose of the medication is reduced or the administration of the medication is ceased to reduce undesirable side effects of the medication. In another embodiment, the clinical response is an at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold or at least 3-fold reduced risk of medication intolerance when compared to a non-genetically guided dosing.

In an even more preferred embodiment, the clinical response is a combination of increased remission rate, decreased intolerability rate, reduced percentage of patients taking medical absence and a reduced average length of medical absence as described herein.

As used herein the term “CNS-active medicament” includes biologically-active compounds that when administered to an individual, elicit a biological response from the CNS. CNS-active medicaments may be stimulants, depressants, anti-depressants, mood stabilisers, etc. Examples of biological response include but are not limited to stimulation or suppression of neuron action potentials or neurological activities.

In one embodiment, the CNS-active medicament is selected from the following the list consisting of sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline. In another preferred embodiment, the CNS-active medicament is desvenlafaxine.

Major Depressive Disorder (MDD) is a severe form of depression and it may be diagnosed by various criteria. The most widely used criteria for diagnosing depressive conditions are found in the American Psychiatric Association's revised fourth edition of the Diagnostic and Statistical Manual of Mental Disorders (DSM-IV-TR), and the World Health Organization's International Statistical Classification of Diseases and Related Health Problems (ICD-10), which uses the name depressive episode for a single episode and recurrent depressive disorder for repeated episodes. The DSM-IV-TR recognises five different subtypes of MDD and these are melancholic depression, atypical depression, catatonic depression, postpartum depression and seasonal affective disorder. As used herein the term “Major Depressive Disorder” (MDD) includes melancholic depression, atypical depression, catatonic depression, postpartum depression and seasonal affective disorder.

Cyclothymic disorder is a mental disorder. It is a mild form of bipolar disorder (manic depressive illness), in which a person has mood swings over a period of years that go from mild depression to emotional highs. The causes of cyclothymic disorder are unknown. Common symptoms of cyclothymic disorder may include periods (episodes) of extreme happiness and high activity or energy (mania), or low mood, activity, or energy (depression) for at least 2 years (1 or more years in children and adolescents), mood swings, ongoing symptoms, with no more than 2 symptom-free months in a row. Treatments for this disorder include mood-stabilizing medicine, antidepressants, talk therapy, or some combination of these three treatments.

Persistent depressive disorder (PDD) is a chronic depression in which a person's moods are regularly low. PDD is also known as dysthymia. The cause of PDD is also unknown. The main symptom of PDD is a low, dark, or sad mood on most days for at least 2 years. In children and teens, the mood can be irritable instead of depressed and lasts for at least 1 year. Other clinical symptoms may include at least one of the following: feelings of hopelessness, too little or too much sleep, low energy or fatigue, low self-esteem, poor appetite or overeating and poor concentration. PDD is also known as chronic depression. Epilepsy is a brain disorder that causes people to have recurring seizures. The seizures happen when clusters of nerve cells, or neurons, in the brain send out the wrong signals. The symptoms may range from the individual experiencing strange sensations and emotions, behaving strangely to violent muscle spasms or losing consciousness. Epilepsy may be caused by brain injury, abnormal brain development, or other unknown causes. There is currently no cure for epilepsy, however medications such as Lamotrigine (6-(2,3-dichlorophenyl)-1,2,4-triazine-3,5-diamine), Levetiracetam ((S)-2-(2-Oxopyrrolidin-1-yl)butanamide), Tiagabine ((−)-(3R)-1-[4,4-bis(3-methyl-2-thienyl)-3-buten-1-yl]-3-piperidinecarboxylic acid), Pregabalin (beta-isobutyl-GABA) have been used to treat seizures.

Schizophrenia is a severe brain disorder in which people interpret reality abnormally. Schizophrenia may result in some combination of hallucinations, delusions, and extremely disordered thinking and behaviour. Drugs for treating schizophrenia includes Aripiprazole, Aripiprazole lauroxil, Fluphenazine, Haloperidol, Olanzapine pamoate, Paliperidone or Risperidone.

Bipolar disorder is the name used to describe a set of ‘mood swing’ conditions, the most severe form of which used to be called ‘manic depression’. Bipolar disorder is classified into type I and type II. Bipolar disorder I is the more severe disorder in terms of symptoms, with individuals being more likely to experience mania, have longer ‘highs’, be more likely to have psychotic experiences and be more likely to be hospitalised. Bipolar disorder II is diagnosed when a person experiences the symptoms of a high but with no psychotic experiences. These hypomanic episodes tending to last a few hours or a few days, but longitudinal studies suggest impairment is often as severe as in bipolar I disorder. The high moods are called mania or hypomania and the low mood is called depression. The cause of bipolar disorder is poorly understood, it is thought that both environmental and genetic factors contribute to the disease. Examples of drugs for treating bipolar disorder includes lithium, lithium carbonate, ketamine, anticonvulsants, carbamazepine, sodium valproate, Lamotrigine, topiramate, antipsychotics, Olanzapine.

Alzheimer's disease is an irreversible, chronic neurodegenerative disease that results in the loss of memory, lose of language abilities, disorientation, mood swings, loss of motivation, and other behavioural issues. There are currently two main classes of drugs for treating Alzheimer's Disease, acetylcholinesterase inhibitors such as tacrine, rivastigmine, galantamine and donepezil and N-mythyl-D-aspartate receptor antagonists such as ketamine, dextromethorphan (DXM), phencyclidine (PCP) and methoxetamine (MXE).

In one embodiment, the present invention provides a method of determining a prognosis of a clinical response to a CNS-active medicament in a patient suffering from bipolar disorder, epilepsy, Schizophrenia and Alzheimer's Disease as the metabolism and pharmacokinetics of the drugs used to treat bipolar disorder, epilepsy, Schizophrenia and Alzheimer's Disease.

As used herein the terms “allele” and “allelic variant” refer to one of more than one alternative forms of the same gene or same genetic locus. The alternative form refers to differences in the DNA sequences. Single Nucleotide Polymorphisms (SNPs) are the most common type of genetic variation among people. Each SNP represents a difference in a single DNA building block, called a nucleotide. For example, a SNP may replace the nucleotide cytosine (C) with the nucleotide thymine (T) in a certain stretch of DNA. A SNP does not necessarily change the encoded amino acid due to degeneracy of the genetic code. SNPs in coding region of the genome are classified as synonymous and non-synonymous depending based on whether the SNP results in a change in the encoded amino acid. As discussed, most SNPs have no effect on health or development. Some of these genetic differences, however, have proven to be important in the study of human health. Researchers have found SNPs that may help predict an individual's response to certain drugs, susceptibility to environmental factors such as toxins, and risk of developing particular diseases. SNPs can also be used to track the inheritance of disease genes within families.

An officially registered SNP is given a Reference SNP identifier number (as denoted by the rs prefix followed by a unique number) by the United States of America, National Institute of Health SNP database dbSNP. Rs identifiers are used herein to refer to SNP genotypes.

By standard convention, the locations of SNPs are based on their chromosomal position. This number changes every time a new reference human genome assembly is released; the current “build” is GRCh38, and the previous one, which is still used by many sources for a variety of reasons, is GRCh37 (also known as hg19).

Genes are read (transcribed) in either the forward or reverse direction, as numbered along the chromosome. If a new build comes along that flips a large segment of a chromosome, the gene direction will change. As a result, at different times, as well as in different publications or different databases, the same SNP can be defined as being on the forward (plus) or as being on the reverse (minus) strand. In terms of the nucleotides for that SNP, the pairing of A with T, and C with G, in the DNA double helix means that an A on the plus strand by definition is a T on the minus strand, and vice versa, and a C on the plus strand means a G on the minus strand (and vice versa). The way a SNP is defined is based on its flanking sequences, the major and minor alleles of a SNP should not change between builds, however, the position number along the chromosome will change, and whether it is on the plus or minus strand may change. Accordingly, if a given genotype does not appear to correspond to the known genotypes of a SNP, it is possible to determine the correct genotype by reference to the specific reference SNP identifier number of the SNP.

Where SNP genotypes are provided in this specification, the genotype will be provided with the orientation of the DNA strand (plus or minus) for clarity. The genotypes provided herein are provided as a pair of SNPs inherited at a given chromosomal position, i.e. one inherited paternally and one inherited maternally. For example, the genotype TT indicates that the individual has a homozygous genotype for this SNP, and the genotype AT indicates that the individual has a heterozygous genotype for this SNP.

The exact mechanism(s) of action of antidepressants remains elusive. Uncertainty about the mechanism of action of antidepressants impedes the identification of clinically useful pharmacodynamic pharmacogenetic treatment biomarkers. Furthermore, human body ‘defences’ make it difficult for xenobiotics (including antidepressants) to reach the pristine cerebrospinal fluid (CSF) bathing the CNS. Inter-individual variations may underscore the different antidepressant doses needed for patients to enjoy tolerable efficacy.

Without wishing to be bound by theory, there is evidence suggesting that Caucasian/carriers at the serotonin transporter linked promoter region (5HTTLPR) are more likely to respond to antidepressants, but the effect size appears too small for clinical utility. The association also appears to be modulated by environmental stress, putatively via epigenetic mechanisms.

In contrast, the present inventor has demonstrated that the presence or absence of an allelic variant of ABCC1 can be used to determine a prognosis of a clinical response, including remission, to antidepressants in patients suffering Major Depressive Disorder. As used herein the term ‘ABCC1’ refers to ATP-binding cassette, sub-family C member 1 (also known as ABCC1, MRP, ABCC GS-X, MRP1 and ABC29). ABCC1 is a member of the superfamily of ATP-binding cassette (ABC) transporters, which transport various molecules across extra- and intra-cellular membranes. This transporter is a multispecific organic anion transporter, with oxidized glutatione, cysteinyl leukotrienes, and activated aflatoxin B1 as substrates. This protein also transports glucuronides and sulfate conjugates of steroid hormones and bile salts. Without wishing to be bound by theory, ABCC1 is considered to be an abluminal transporter expressed at the blood brain barrier.

An exemplary partial sequence of ABCC1 showing the nucleotides surrounding the rs212090 SNP (the nucleotide at the SNP position is underlined and in bold), is shown in SEQ ID NO: 1 and reproduced below as follows:

| | |
|---|---|
| 1 | GACCAAATTC AGCCTACTGC CTCGGATCTC TCCAGCCGAA GTCTGTGGAC TGCAAGTCTT |
| 61 | TGAGATGCTT CTGGCTCCCA TCACCTCTAA CATCCTTGTC TGGGTCTACC AGGAACGCTT |
| 121 | CATTTCCTTG GGGCTGCAGT TTTGTGGTTG AGGGGCCTGG AGAAAATCAT TTTCTCCCCT |
| 181 | TGGCAGTGTC CCAGGGCCCT GGATGGTCCT CTTACCAACA TCTGGTCTTC CAGGCACTCA |
| 241 | AAAGCTGGGA ACCAGCATCT CAGCGCCAGC TCTACCAGTT CTCGTTTTGG GCCAGAGGCA |
| 301 | GCCTCTGCAC TCCCACGCCT GTCCTCCTGG AAGGGACCTG GTTGGACTAA CGGCTAACCT |
| 361 | GGACCTGGAA CTGTAGGGCC AGGGGATTGT CTCAGGGCCG ACGTTCCACC TGGGGCTTCC |
| 421 | CTCCCCACCC ACCCCGACTC CAGGCTTTCC CTTTTTTCTT TTGTTCAACA TTGTAAGAAC |
| 481 | AATCAATGCT GTTATTACTG TTCCCACCAT GATTGATGTG GGGTAAATAT TAAGGAGATG |
| 541 | GCCTCATGGG AATTTGACCT TGACTAGAAA TAGAGACTGA GAGTGAGCAA CCAGCTGGAA |
| 601 | GGTACTATGC CAGTCCTAGC AGAAAAATGT GTTAGGGGCC TGGCCCAAAG CAGTGTTGGT |
| 661 | TGCTTACAGT GTTGATTGAT TTTGTTCTTT TTTCTTACCA CCTCTTTTCT TTCCCTCTCA |
| 721 | TGGTACCTGC TCATGGTTAT GAAGCTTTCA AAGTAAAGAA CACGAAATAC CTCCCAAGTA |
| 781 | TTACCAGTGG GTACCAAAAA AATGTCCCCT TGAGTCTTTT CCTTGTTTTT AGATGTTAAT |
| 841 | TCTCTCCCTT GGCATCCGGT TAGCCCCCCA GGGGGGGCAG CATTGTGGAG AACTTGATAT |
| 901 | TTAGTTACTG ATGCTCTTCC AGGACACGAA AAGAACCCAT CTTTGAATAT CAATGATTTT |
| 961 | TTTTTTTTAA GTACTGTTCC GGGGAGAAAA ACAGTCTCAA A |

As used herein the term “determining”, when used to refer to the presence or absence of an allelic variant or an allele, includes identifying the nucleic acid sequence (i.e. A, G, C or T) of the genomic region relevant to the allele, and comparing the identified sequence with the reference sequence to identify any differences in the nucleic acid sequence. There are a number of ways to determine the nucleic acid sequence of a deoxyribonucleic acid (DNA) molecule such as Sanger sequencing, shotgun sequencing, bridge PCR, next-generation sequencing, 454 sequencing, Illumina sequencing, Ion Torrent, SOLiD, pyrosequencing, etc. Any sequencing method capable of distinguishing single nucleotide variations is appropriate.

As used herein, the term “presence of an allelic variant” includes a particular allelic variant being present in a heterozygous or a homozygous state in the genotype of a patient.

As used herein, the term “absence of an allelic variant” refers to a particular allelic variant being absent in the genotype of a patient. The absence of an allelic variant in an individual may indicate that the individual is wildtype at that locus.

As used herein the term “improved clinical response” includes a desired change in the patent's response to the administered CNS-active medicament. In the context of the present invention, improved clinical response may be assessed using the same methods as the original MDD diagnosis and improved clinical response may be measured by clinical scoring etc. For example, an improved clinical response includes an increased pharmacologic response to a CNS-active medicament, improved treatment of one or more symptoms associated with major depressive disorder, cyclothymic disorder, or persistent depressive disorder (e.g. a reduction of the severity of a symptom or the frequency of occurrence of a symptom of major depressive disorder, cyclothymic disorder, or persistent depressive disorder), increased remission, decreased medical absence and decreased intolerance to the CNS-active medicament.

As used herein the term “worsened clinical response” includes no change or an undesirable change in the patent's response to the administered CNS-active medicament. In the context of the present invention, worsened clinical response may be assessed using the same methods as the original MDD diagnosis and worsened clinical response may be measured by clinical scoring etc. For example, a worsened clinical response includes an unchanged or decreased pharmacologic response to a CNS-active medicament, unchanged or diminished treatment of one or more symptoms associated with major depressive disorder, cyclothymic disorder, or persistent depressive disorder (e.g. unchanged or increased severity of a symptom or the frequency of occurrence of a symptom of major depressive disorder, cyclothymic disorder, or persistent depressive disorder), unchanged or decreased remission, unchanged or increased medical absence and unchanged or increased intolerance to the CNS-active medicament.

In one embodiment, the increased clinical response in a patent following genetically guided dosing is relative to the level of clinical response in the patient following genetically unguided dosing. In another embodiment, the increased clinical response in a patent following genetically guided dosing is relative to the level of clinical response in another patient following genetically unguided dosing.

In one embodiment, the allelic variant of ABCC1 determined in the method described herein is rs212090, which defines three genotypes A;A (homozygous A), A;T (heterozygous) and T;T (homozygous) (plus orientation, GRCh38). The nucleotide variation is in the 3′ untranslated region of the ABCC1 messenger RNA (mRNA) and appears to influence ABCC1 transcription. The altered transcription mechanism may involve differential mRNA stability. This may have functional effects on the ABCC1 transporter, although the precise biological effect of these SNPs on ABCC1 is not yet understood.

The specific mutations are as follows:
NC_000016.10:g.16142147T>A;
NC_000016.9:g.16236004T>A;
NG_028268.1:g.197571T>A;
NT_187607.1:g.1800038A=; or
NT_187607.1:g.1800038A>T.

The inventor of the present invention has now demonstrated that the presence of an A;A or A;T allelic variant of rs212090 indicates an improved clinical response to the CNS-active medicament. Furthermore, the presence of a T;T allelic variant of rs212090 indicates a worsened clinical response to the CNS-active medicament.

The present inventor has also demonstrated that the presence or absence of an allelic variant of ABCB1 can be used to determine a prognosis of a clinical response, including remission, to antidepressants in patients suffering Major Depressive Disorder. Accordingly, in one embodiment, the present invention provides a method of determining a prognosis of a clinical response to a CNS-active medicament in a patient suffering from major depressive disorder, cyclothymic disorder, or persistent depressive disorder, said method comprising determining the presence or absence of an allelic variant of ABCC1; wherein the presence or absence the allelic variant indicates an improved or worsened clinical response to the CNS-active medicament, further comprising determining the presence or absence of an allelic variant of ABCB1.

Without wishing to be bound by theory, the ABCB1 transporter was discovered during study of chemotherapeutic resistance in hamster cancer cells. It is now known to be a member of a larger ATP-binding cassette family of transporter proteins involved in multidrug resistance. This family of transporters appears to be highly conserved among species ABCB1 has been shown to be evenly expressed and functionally protective across the entire human brain. It appears ABCB1 has a key role in the CNS bioavailability of several psychotropics. The importance of ABCB1 in drug bioavailability was emphasised using human duodenal cells that showed the T;T polymorphism of the rs1045642 reduced ABCB1 efflux. This is a synonymous single nucleotide variation producing a codon that does not change the expressed amino acid (isoleucine). In a study utilising cultured human HeLa cell lines, T;T genotype at rs1045642 resulted in use of the rarer isoleucine codon (ATT rarer than ATC), altering ABCB1 conformational folding and through this mechanisms altering efflux functioning.

In a further embodiment, the method as herein described further comprises determining the presence or absence of an allelic variant of ABCB1. An exemplary partial sequence of ABCB1 showing the nucleotides surrounding the rs1045642 SNP (the nucleotide at the SNP position is underlined and in bold), is shown in SEQ ID NO: 2 and reproduced below as follows:

| | |
|---|---|
| 1 | ATTAGCAACC TTACATCTAC TACTTTAGTT TCTTTTTGCC ATGTAACATA ACACATTCAC |
| 61 | AGGATCCAGG GATTAGGACA CAGATGTCTT GTGGGAGAGG GAACATTATT CTGCCTACCA |
| 121 | CATGCATACA TCAGAAACCA TGGTTGAAAC ACAGGAAACA TGACAGTTCC TCAAGGCATA |
| 181 | CAATTATGAC CTTGTTGGGT TAACCTTCAC TATCCAAATT TTAATCACAC AAACTTTTCC |
| 241 | TTAATCTCAC AGTAACTTGG CAGTTTCAGT GTAAGAAATA ATGATGTTAA TTGTGCTACA |
| 301 | TTCAAAGTGT GCTGGTCCTG AAGTTGATCT GTGAACTCTT GTTTTCAGCT GCTTGATGGC |
| 361 | AAAGAAATAA AGCGACTGAA TGTTCAGTGG CTCCGAGCAC ACCTGGGCAT CGTGTCCCAG |
| 421 | GAGCCCATCC TGTTTGACTG CAGCATTGCT GAGAACATTG CCTATGGAGA CAACAGCCGG |
| 481 | GTGGTGTCAC AGGAAGAGAT CGTGAGGGCA GCAAAGGAGG CCAACATACA TGCCTTCATC |
| 541 | GAGTCACTGC CTAATGTAAG TCTCTCTTCA AATAAACAGC CTGGGAGCAT GTGGCAGCCT |
| 601 | CTCTGGCCTA TAGTTTGATT TATAAGGGGC TGGTCTCCCA GAAGTGAAGA GAAATTAGCA |
| 661 | ACCAAATCAC ACCCTTACCT GTATACAAGC ATCTGGCCAC ACTTCCTGTT TGGGTTAGTT |
| 721 | GTTACCTTTA CCTGATCACC TGACCCTCCT TGTGAGGAAG GGATGAAAGT GTTCGACCAC |
| 781 | TTCAGGTTTA GGAGAGAGGA ACATTTCTGG GATAGGAGAA CTGGAACAAT TGTCTTGATC |
| 841 | CAAAGCTATA GGCTTGAGGC TCCACCTTTG TCAGCCTTAG GGGTAAGTAC AATATCTGGA |
| 901 | AAGCCTTTCA CTTTAAGTCC AAGTACAGAG TCTGGGTCCC CACCTGCACA TGCTGCTTCT |
| 961 | GGCCTGCTGA GGAAGTAGGC ATGACTGTCT CTCCCCATGT C |

In an embodiment, the allelic variant of ABCB1 determined in the method described herein is rs1045642, which defines three genotypes C;C, T;C and T;T (minus strand, GRCh38). The specific mutations are as follows:
NC_000007.13:g.87138645A>G;
NC_000007.14:g.87509329A>G; or
NG_011513.1:g.208920T>C.

The inventor of the present invention has now demonstrated that the presence of a T;T allelic variant of rs1045642 indicates an improved clinical response to a CNS-active medicament. Furthermore, the presence of a C;C or C;T allelic variant of rs1045642 is demonstrated herein to indicate a worsened clinical response to a CNS-active medicaments.

Example 3 also demonstrates rs212090 (ABCC1) and rs1045642 (ABCB1) can be used in combination to determine a prognosis of a clinical response to desvenlafaxine in a patient suffering from MDD. Example 3 also demonstrates rs212090 (ABCC1), rs1045642 (ABCB1) and rs8175347 (UGT1A1) can be used in combination to determine a prognosis of a clinical response to desvenlafaxine in a patient suffering from MDD.

In one embodiment, the one or more allelic variants detected are the rs212090 allelic variant of ABCC1 and the rs1045642 allelic variant of ABCB1.

In another embodiment, the one or more allelic variants detected are the rs212090 allelic variant of ABCC1, the rs1045642 allelic variant of ABCB1 and the rs8175347 and rs4148323 allelic variant of UGT1A1.

ABCB1 appears to be expressed on the blood (luminal) side of the blood brain barrier endothelia, whereas ABCC1 appears to be expressed on the CSF (abluminal) side of the BBB. It appears that endogenous inhibitors of ABCB1 are transported into BBB endothelia by ABCC1. Without wishing to be bound by theory, ABCB1 and ABCC1 are thought to have similar substrate affinities, so should a toxin impede the function of both transporters, the reduced functioning of ABCC1 may result in reduced transport by ABCC1 of ABCB1 endogenous inhibitors, enabling greater activity of ABCB1 in real time to defend the CNS against toxins. Accordingly, an allelic variant of ABCC1 gene may lead to reduced expression of ABCC1 and therefore greater ABCB1 activity, and greater pharmacokinetic blockade at the BBB of CNS-active medicaments.

Without wishing to be bound by theory, failure of previous pharmacogenetic studies to control for the influence of phase I hepatic metabolism genotype, phase II hepatic metabolism genotype, ABCB1 transporter genotype, and ABCC1 transporter genotype may explain the mixed findings in the literature to date—which has mainly consisted of association studies. In contrast, the present inventor has also demonstrated that hepatic metabolism genotypes can surprisingly increase the sensitivity and specificity of methods of determining a prognosis of a clinical response to a CNS active medicament. For example, the presence or absence of an allelic variant of UGT1A1, or UGT1A1, CYP2D6 and CYP2C19, in combination with ABCC1 and ABCB1 can be used to determine a prognosis of a clinical response, including remission, to antidepressants in patients suffering Major Depressive Disorder.

Accordingly, in one embodiment, the present invention provides a method of determining a prognosis of a clinical response to a CNS-active medicament in a patient suffering from major depressive disorder, cyclothymic disorder, or persistent depressive disorder, said method comprising determining the presence or absence of an allelic variant of ABCC1; wherein the presence or absence the allelic variant indicates an improved or worsened clinical response to the CNS-active medicament, further comprising determining the presence or absence of an allelic variant of ABCB1, and further comprising determining the presence or absence of an allelic variant selected from the group consisting of CYP2D6, CYP2C19 and UGT1A1.

CYP2D6 encodes a cytochrome P450 enzyme, which is important in the metabolism of xenobiotics in the human body. It is mostly expressed in the liver and the central nervous system. An exemplary partial sequence of CYP2D6 showing the nucleotides surrounding the rs3892097 SNP (the ancestral nucleotide at the SNP position is underlined and in bold), is shown in SEQ ID NO: 3, and reproduced below as follows:

| | |
|---|---|
| 1 | CTGGACAGAG CCAGGGACTG CGGGAGACCA GGGGGAGCAT AGGGTTGGAG TGGGTGGTGG |
| 61 | ATGGTGGGGC TAATGCCTTC ATGGCCACGC GCACGTGCCC GTCCCACCCC CAGGGGTGTT |
| 121 | CCTGGCGCGC TATGGGCCCG CGTGGCGCGA GCAGAGGCGC TTCTCCGTCT CCACCTTGCG |
| 181 | CAACTTGGGC CTGGGCAAGA AGTCGCTGGA GCAGTGGGTG ACCGAGGAGG CCGCCTGCCT |
| 241 | TTGTGCCGCC TTCGCCAACC ACTCCGGTGG GTGATGGGCA GAAGGGCACA AAGCGGGAAC |
| 301 | TGGGAAGGCG GGGGACGGGG AAGGCGACCC CTTACCCGCA TCTCCCACCC CCAGGACGCC |
| 361 | CCTTTCGCCC CAACGGTCTC TTGGACAAAG CCGTGAGCAA CGTGATCGCC TCCCTCACCT |
| 421 | GCGGGCGCCG CTTCGAGTAC GACGACCCTC GCTTCCTCAG GCTGCTGGAC CTAGCTCAGG |
| 481 | AGGGACTGAA GGAGGAGTCG GGCTTTCTGC GCGAGGTGCG GAGCGAGAGA CCGAGGAGTC |
| 541 | TCTGCAGGGC GAGCTCCCGA GAGGTGCCGG GGCTGGACTG GAGCCTCGGA AGACAGGATT |
| 601 | TGCATAGATG GGTTTGGGAA AGACATTCCA GAGACCCCAC TGTAAGAGGG CCTGGAGAGG |
| 661 | AGG |

An exemplary partial sequence of CYP2D6 showing the nucleotides surrounding the rs1065852 SNP (the ancestral nucleotide at the SNP position is underlined and in bold), is shown in SEQ ID NO: 4, and reproduced below as follows:

| | |
|---|---|
| 1 | TTAGGGTGTA TGAGCCTAGC TGGGAGGTGG ATGGCCGGGT CCACTGAAAC CCTGGTTATC |
| 61 | CCAGAAGGCT TTGCAGGCTT CAGGAGCTTG GAGTGGGGAG AGGGGGTGAC TTCTCCGACC |
| 121 | AGGCCCCTCC ACCGGCCTAC CCTGGGTAAG GGCCTGGAGC AGGAAGCAGG GGCAAGAACC |
| 181 | TCTGGAGCAG CCCATACCCG CCCTGGCCTG ACTCTGCCAC TGGCAGCACA GTCAACACAG |
| 241 | CAGGTTCACT CACAGCAGAG GGCAAAGGCC ATCATCAGCT CCCTTTATAA GGGAAGGGTC |
| 301 | ACGCGCTCGG TGTGCTGAGA GTGTCCTGCC TGGTCCTCTG TGCCTGGTGG GGTGGGGGTG |
| 361 | CCAGGTGTGT CCAGAGGAGC CCATTTGGTA GTGAGGCAGG TATGGGGCTA GAAGCACTGG |
| 421 | TGCCCCTGGC CGTGATAGTG GCCATCTTCC TGCTCCTGGT GGACCTGATG CACCGGCGCC |
| 481 | AACGCTGGGC TGCACGCTAC CCACCAGGCC CCCTGCCACT GCCCGGGCTG GGCAACCTGC |
| 541 | TGCATGTGGA CTTCCAGAAC ACACCATACT GCTTCGACCA GGTGAGGGAG GAGGTCCTGG |
| 601 | AGGGCGGCAG AGGTCCTGAG GATGCCCCAC CACCAGCAAA CATGGGTGGT GGGTGAAACC |
| 661 | ACAGGCTGGA CCAGAAGCCA GGCTGAGAAG GGGAAGCAGG TTTGGGGGAC TTCCTGGAGA |
| 721 | AGGGCATTTA TACATGGCAT GAAGGACTGG ATTTTCCAAA GGCCAAGGAA GAGTAGGGCA |
| 781 | AGGGCCTGGA GGTGGAGCTG GACTTGGCAG TGGGCATGCA AGCCCATTGG GCAACATATG |
| 841 | TTATGGAGTA CAAAGTCCCT TCTGCTGACA CCAGAAGGAA AGGCCTTGGG AATGGAAGAT |
| 901 | GAGTTAGTCC TGAGTGCCGT TTAAATCACG AAATCGAGGA TGAAGGGGGT GCAGTGACCC |
| 961 | GGTTCAAACC TTTTGCACTG TGGGTCCTCG GGCCTCACTG C |

An exemplary partial sequence of CYP2D6 showing the nucleotides surrounding the rs28371725 SNP (the ancestral nucleotide at the SNP position is underlined and in bold), is shown in SEQ ID NO: 5, and reproduced below as follows:

| | |
|---|---|
| 1 | CAAGGTCCTA CGCTTCCAAA AGGCTTTCCT GACCCAGCTG GATGAGCTGC TAACTGAGCA |
| 61 | CAGGATGACC TGGGACCCAG CCCAGCCCCC CCGAGACCTG ACTGAGGCCT TCCTGGCAGA |
| 121 | GATGGAGAAG GTGAGAGTGG CTGCCACGGT GGGGGGCAAG GGTGGTGGGT TGAGCGTCCC |
| 181 | AGGAGGAATG AGGGGAGGCT GGGCAAAAGG TTGGACCAGT GCATCACCCG GCGAGCCGCA |
| 241 | TCTGGGCTGA CAGGTGCAGA ATTGGAGGTC ATTTGGGGGC TACCCCGTTC TGTCCCGAGT |
| 301 | ATGCTCTCGG CCCTGCTCAG GCCAAGGGGA ACCCTGAGAG CAGCTTCAAT GATGAGAACC |
| 361 | TGTGCATAGT GGTGGCTGAC CTGTTCTCTG CCGGGATGGT GACCACCTCG ACCACGCTGG |
| 421 | CCTGGGGCCT CCTGCTCATG ATCCTACATC CGGATGTGCA GCGTGAGCCC ATCTGGGAAA |
| 481 | CAGTGCAGGG GCCGAGGGAG GAAGGGTACA GGCGGGGGCC CATGAACTTT GCTGGGACAC |
| 541 | CCGGGGCTCC AAGCACAGGC TTGACCAGGA TCCTGTAAGC CTGACCTCCT CCAACATAGG |
| 601 | AGGCAAGAAG GAGTGTCAGG GCCGGACCCC CTGGGTGCTG ACCCATTGTG GGGACGCATG |
| 661 | TCTGTCCAGG CCGTGTCCAA CAGGAGATCG ACGACGTGAT AGGGCAGGTG CGGCGACCAG |
| 721 | AGATGGGTGA CCAGGCTCAC ATGCCCTACA CCACTGCCGT GATTCATGAG GTGCAGCGCT |
| 781 | TTGGGGACAT CGTCCCCCTG GGTGTGACCC ATATGACATC CCGTGACATC GAAGTACAGG |
| 841 | GCTTCCGCAT CCCTAAGGTA GGCCTGGCGC CCTCCTCACC CCAGCTCAGC ACCAGCCCCT |
| 901 | GGTGATAGCC CCAGCATGGC TACTGCCAGG TGGGCCCACT CTAGGAACCC TGGCCACCTA |
| 961 | GTCCTCAATG CCACCACACT GACTGTCCCC ACTTGGGTGG G |

CYP2C19 encodes a member of the cytochrome P450 superfamily of enzymes. The cytochrome P450 proteins are monooxygenases, which catalyse many reactions involved in drug metabolism and synthesis of cholesterol, steroids and other lipids. An exemplary partial sequence of CYP2C19 showing the nucleotides surrounding the rs4244285 SNP (the nucleotide at the SNP position is underlined and in bold), is shown in SEQ ID NO: 6 and reproduced below as follows:

| | |
|---|---|
| 1 | CTCCCACTTC TAAAATACTA ATTCAATTTC AGAGGCTGCT TGATAGAAAT CAATATAGCA |
| 61 | GGGACTATCT TTGTAGTATC AATCAGGTTG TGCAAACTCT TTTAACCTAT GCTATCATCT |
| 121 | CCAAAATGTT AATGTAGTAA TTCATACCAT CTTATATTTC AAGATTGTAG AGAAGAATTG |
| 181 | TTGTAAAAAG TAAGAGAATT AATATAAAGA TGCTTTTATA CTATCAAAAG CAGGTATAAG |
| 241 | TCTAGGAAAT GATTATCATC TTTGATTCTC TTGTCAGAAT TTTCTTTCTC AAATCTTGTA |
| 301 | TAATCAGAGA ATTACTACAC ATGTACAATA AAAATTTCCC CATCAAGATA TACAATATAT |
| 361 | TTTATTTATA TTTATAGTTT TAAATTACAA CCAGAGCTTG GCATATTGTA TCTATACCTT |
| 421 | TATTAAATGC TTTTAATTTA ATAAATTATT GTTTTCTCTT AGATATGCAA TAATTTTCCC |
| 481 | ACTATCATTG ATTATTTCCC GGGAACCCAT AACAAATTAC TTAAAAACCT TGCTTTTATG |
| 541 | GAAAGTGATA TTTTGGAGAA AGTAAAAGAA CACCAAGAAT CGATGGACAT CAACAACCCT |
| 601 | CGGGACTTTA TTGATTGCTT CCTGATCAAA ATGGAGAAGG TAAAATGTTA ACAAAAGCTT |
| 661 | AGTTATGTGA CTGCTTGCGT ATTTGTGATT CATTGACTAG TTTTGTGTTT ACTACGGATG |
| 721 | TTTAACAGGT CAAGGAGTAA TGCTTGAGAA GCATATTTAA GTTTTTATTG TATGCATGAA |
| 781 | TATCCAGTAA GCATCATAGA AAATGTAAAA TTAAATTGTT AAATAATTAG AATACATAGA |
| 841 | AGAAATTGTT TAGATAAATA TAATCTATCT GAACAATAAG GATGTCAGGA TAGGAAAAGC |
| 901 | TCTGTTCTGC AGCTTCCAGT GAGATCAGCA CAGGAGGAAC TTAAATTTAA AAGAAAATAA |
| 961 | AAAACATCTC CATCAAAAAG TGAGTGAAGG ATATGAACAG A |

An exemplary partial sequence of CYP2C19 showing the nucleotides surrounding the rs4986893 SNP (the ancestral nucleotide at the SNP position is underlined and in bold), is shown in SEQ ID NO: 7, and reproduced below as follows:

| | |
|---|---|
| 1 | TTGTCTACAG ACTTTGCAGA CTGATGTGAT TCCCTCTGAA ACTTGAATTA TTTGGTTTCT |
| 61 | AAAAAAGTCT CTTTTTTTCT TTCCAAAGTA AAAGACAAAT AGGCCGGGAA TGTAAATTTA |
| 121 | GCATTTGAGC AACCATTATT TAACCAGCTA GGCTGTAATT GTTAATTCGA GATTAATGTA |
| 181 | AAAGTGATGT GTTGATTTTA TGCATGCCAA ACTCTTTTTT GCTTTTAAGG GAATTCATAG |
| 241 | GTAAGATATT ACTTAAAATT TCTAAACTAT TATTATCTGT TAACAAATAT GAAGTGTTTT |
| 301 | ATATCTAATG TTTACTCATA TTTTAAAATT GTTTCCAATC ATTTAGCTTC ACCCTGTGAT |
| 361 | CCCACTTTCA TCCTGGGCTG TGCTCCCTGC AATGTGATCT GCTCCATTAT TTTCCAGAAA |
| 421 | CGTTTCGATT ATAAAGATCA GCAATTTCTT AACTTGATGG AAAAATTGAA TGAAAACATC |
| 481 | AGGATTGTAA GCACCCCCTG GATCCAGGTA AGGCCAAGTT TTTTGCTTCC TGAGAAACCA |
| 541 | CTTACAGTCT TTTTTTCTGG GAAATCCAAA ATTCTATATT GACCAAGCCC TGAAGTACAT |
| 601 | TTTTGAATAC TACAGTCTTG CCTAGACAGC CATGGGGTGA ATATCTGGAA AAGATGGCAA |
| 661 | AGTTCTTTAT TTTATGCACA GGAAATGAAT ATCCCAATAT AGATCAGGCT TCTAAGCCCA |
| 721 | TTAGCTCCCT GATCAGTGTT TTTTCCACTA AACTCCAAAG CCCTGTTTCT ATAAAGTACT |
| 781 | TTGGTGACAG CCCCAAAGCG TGCTTATATC ACTCCATGGA CATCCAGGCA CTTTGGAGTC |
| 841 | TTCCATTACT CACAAGGCTT GTCCTTCAAT TCACACTTTG TCATATTGTG TGACAGAAAT |
| 901 | ATCCTAATCT AAAAGACATT ATCTCCTTCA AGGACAGAGA ATATTTGGAA CCACAGAAGC |
| 961 | TGCCAAGAAA CACTGAATAG GGCAGAGGTG TTTGATGTCT C |

An exemplary partial sequence of CYP2C19 showing the nucleotides surrounding the rs12248560 SNP (the ancestral nucleotide at the SNP position is underlined and in bold), is shown in SEQ ID NO: 8, and reproduced below as follows:

| | |
|---|---|
| 1 | GCTCCCGTTA AGGTCTATAC ATTGTGGTGG TTTTGTGCTG TGGGTCCATT TAGTGATTTC |
| 61 | CCTACCTCCC ATCCTCTATT AGATTCACAA CTGTTGTTCT GCCCATAATT TCCTATGCTT |
| 121 | GCTTTGCATT GTTACATTTT TTTTTGAAAA TCAGAAAGCA AAATCAATAT AAAGCAGCCA |
| 181 | TGTCTGGAGG AGACCAGGAG GTCAAGAAGC CTTAGTTTCT CAAGCCCTTA GCACCAAATT |
| 241 | CTCTGAGATC AGCTCTTCCT TCAGTTACAC TGAGCATTTC CCCTCTGCAG TGATGGAGAA |
| 301 | GGGAGAACTC TTATTTTTTC TCATGAGCAT CTCTGGGGCT GTTTTCCTTA GATAAATAAG |
| 361 | TGGTTCTATT TAATGTGAAG CCTGTTTTAT GAACAGGATG AATGTGGTAT ATATTCAGAA |
| 421 | TAACTAATGT TTGGAAGTTG TTTTGTTTTG CTAAAACAAA GTTTTAGCAA ACGATTTTTT |
| 481 | TTTTCAAATT TGTGTCTTCT GTTCTCAAAG CATCTCTGAT GTAAGAGATA ATGCGCCACG |
| 541 | ATGGGCATCA GAAGACCTCA GCTCAAATCC CAGTTCTGCC AGCTATGAGC TGTGTGGCAC |
| 601 | CAACAGGTGT CCTGTTCTCC CAGGGTCTCC CTTTTCCCAT TTGAAATATA AAAAATAACA |
| 661 | ATTCCTGCCT TCACGTGTTT TTTTAGGGGG TTAAATGGTA AAGGTGTTTA TATCTGCTAA |
| 721 | GGTAATTTAC TTGATATATG TTTGGTTATT GAAGATATAT GAGTTATGTT AGCTATTTCA |
| 781 | TGTTTAGGCT GCTGTATTTT TAGTAGGCTA TATTAAATAG AGGATTTCAT TATAAAGGAC |
| 841 | AAAGTCTCCT AATCTTCGAT ATAGGATTGA CATACTTTTT AAATATACAA GGCATAGAAT |
| 901 | ATGGCCATTT CCGTTAAATC ATAAATTCCC AACTGGTTAT TAATCTAAGA ATTCAGAATT |
| 961 | TTAAGTAATT GTTTTTGCAT CAGATTGTTT ACTTCAGTGC TCTCAATTAT GACGGTGCAT |
| 1021 | TGGAACCACT TGGGTTAACA TTTTTTTGTT TTTATTACCA ATACCTAGGC TTCAACCTAG |
| 1081 | TACAATGAAA |

UGT1A1 gene encodes a UDP-glucuronosyltransferase, an enzyme of the glucuronidation pathway that transforms small lipophilic molecules, such as steroids, bilirubin, hormones, and drugs, into water-soluble, excretable metabolites. An exemplary partial sequence of UGT1A1 showing the nucleotides surrounding the rs8175347 SNP (SNP nucleotide position is indicated as “N” and underlined and in bold), is shown in SEQ ID NO: 9 and reproduced below as follows:

| | |
|---|---|
| 1 | TTTGTGGACT GACAGCTTTT TATAGTCACG TGACACAGTC AAACATTAAC TTGGTGTATC |
| 61 | GATTGGTTTT TGCCANAGTA GGAGAGGGCG AACCTCTGGC AGGAGCAAAG GCGCCATGGC |
| 121 | TGTGGAGTCC CAGGGCGGAC GCCCACTTGT C |

An exemplary partial sequence of UGT1A1 showing the nucleotides surrounding the rs4148323 SNP (the ancestral nucleotide at the SNP position is underlined and in bold), is shown in SEQ ID NO: 10 and reproduced below as follows:

| | |
|---|---|
| 1 | AATACTAATT TAATGGATCC TGAGGTTCTG GAAGTACTTT GCTGTGTTCA CTCAAGAATG |
| 61 | TGATTTGAGT ATGAAATTCC AGCCAGTTCA ACTGTTGTTG CCTATTAAGA AACCTAATAA |
| 121 | AGCTCCACCT TCTTTATCTC TGAAAGTGAA CTCCCTGCTA CCTTTGTGGA CTGACAGCTT |
| 181 | TTTATAGTCA CGTGACACAG TCAAACATTA ACTTGGTGTA TCGATTGGTT TTTGCCATAT |
| 241 | ATATATATAT AAGTAGGAGA GGGCGAACCT CTGGCAGGAG CAAAGGCGCC ATGGCTGTGG |
| 301 | AGTCCCAGGG CGGACGCCCA CTTGTCCTGG GCCTGCTGCT GTGTGTGCTG GGCCCAGTGG |
| 361 | TGTCCCATGC TGGGAAGATA CTGTTGATCC CAGTGGATGG CAGCCACTGG CTGAGCATGC |
| 421 | TTGGGGCCAT CCAGCAGCTG CAGCAGAGGG GACATGAAAT AGTTGTCCTA GCACCTGACG |
| 481 | CCTCGTTGTA CATCAGAGAC GGAGCATTTT ACACCTTGAA GACGTACCCT GTGCCATTCC |
| 541 | AAAGGGAGGA TGTGAAAGAG TCTTTTGTTA GTCTCGGGCA TAATGTTTTT GAGAATGATT |
| 601 | CTTTCCTGCA GCGTGTGATC AAAACATACA AGAAAATAAA AAAGGACTCT GCTATGCTTT |
| 661 | TGTCTGGCTG TTCCCACTTA CTGCACAACA AGGAGCTCAT GGCCTCCCTG GCAGAAAGCA |
| 721 | GCTTTGATGT CATGCTGACG GACCCTTTCC TTCCTTGCAG CCCCATCGTG GCCCAGTACC |
| 781 | TGTCTCTGCC CACTGTATTC TTCTTGCATG CACTGCCATG CAGCCTGGAA TTTGAGGCTA |
| 841 | CCCAGTGCCC CAACCCATTC TCCTACGTGC CCAGGCCTCT CTCCTCTCAT TCAGATCACA |
| 901 | TGACCTTCCT GCAGCGGGTG AAGAACATGC TCATTGCCTT TTCACAGAAC TTTCTGTGCG |
| 961 | ACGTGGTTTA TTCCCCGTAT GCAACCCTTG CCTCAGAATT C |

Genetic variation affecting hepatic cytochrome P450 metaboliser status has been demonstrated in CYP2D6 and CYP2C19 enzyme subtypes, which are involved in the metabolism of most second generation antidepressants and commonly show functional variance between individuals. Polymorphisms of CYP450 enzyme genes have been correlated to enzyme activity, enabling phenotype estimation from the related genotype into poor metaboliser (PM), intermediate metaboliser (IM), extensive metaboliser (EM), and more rarely ultrarapid metaboliser (UM). A 30-70% dose reduction for PM patients and a 135-180% dose elevation in UM patients prescribed CYP2D6 and CYP2C19 dependent antidepressants has been proposed. One second generation antidepressant (desvenlafaxine) is not subject to such phase I CYP450 metabolism, however it is subject to metabolism by the phase II hepatic enzyme UGT1A1 for which only 10% of subjects have rapid metaboliser status. One may expect less inter-individual dose variance for desvenlafaxine given this, but the dose range in clinical settings is large.

Without wishing to be bound by theory, the present inventor proposes the inter-individual dose variance for desvenlafaxine may in part be explained by inter-individual differences in BBB transporter systems. Accordingly, in one aspect the present invention provides a method as described herein wherein the CNS active medicament is desvenlafaxine.

In one embodiment, the allelic variant of CYP2D6 analysed in the method as hereinbefore described is one or more of rs3892097, rs1065852, rs28371725 and a deletion or duplication of P450 2D6.

Rs3892097 describes three genotypes, i.e. A;A, A;G and G;G (minus strand, GRCh38). The specific mutations are as follows:
NC_000022.10:g.42524947C=;
NC_000022.10:g.42524947C>T;
NC_000022.11:g.42128945C=;
NC_000022.11:g.42128945C>T;
NG_008376.3:g.6047G=;
NG_008376.3:g.6047G>A;
NT_187682.1:g.51286C=
NT_187682.1:g.51286C>T
NW_004504305.1:g.51272T=
NW_004504305.1:g.51272T>C
NW_009646208.1:g.14511C=; or
NW_009646208.1:g.14511C>T.

Rs1065852 describes three different genotypes, i.e. C;C, C;T and T;T (minus strand, GRCh37). The specific mutations are as follows:
NC_000022.10:g.42526694G=;
NC_000022.10:g.42526694G>A;
NC_000022.11:g.42130692G=;
NC_000022.11:g.42130692G>A;
NG_008376.3:g.4300C=;
NG_008376.3:g.4300C>T;
NT_187682.1:g.53033G=
NT_187682.1:g.53033G>A
NW_004504305.1:g.53019A=
NW_004504305.1:g.53019A>G
NW_009646208.1:g.16258A=; or
NW_009646208.1:g.16258A>G.

Rs28371725 describes three different genotypes, i.e. A;A, A;G and G;G (minus strand, GRCh37). The specific mutations are as follows:
NC_000022.10:g.42523805C>T;
NC_000022.11:g.42127803C>T;
NG_008376.3:g.7189G>A;
NT_187682.1:g.50144C>T;
NW_004504305.1:g.50130C>T; or
NW_009646208.1:g.13369C>T.

In another embodiment, the allelic variant of CYP2C19 analysed in the method as hereinbefore described is one or more of rs4244285, rs4986893 and rs12248560.

Rs4244285 describes three different genotypes, i.e. A;A, A;G and G;G (plus strand, GRCh38). The specific mutations are as follows:
NC_000010.10:g.96541616G>A;
NC_000010.11:g.94781859G>A; or
NG_008384.2:g.24154G>A.

Rs4986893 describes three different genotypes, i.e. A;A, A;G and G;G (plus strand, GRCh38). The specific mutations are as follows:
NC_000010.10:g.96540410G>A
NC_000010.11:g.94780653G>A
NG_008384.2:g.22948G>A

Rs12248560 describes three different genotypes, i.e. C;C, C;T and T;T (plus strand, GRCh38). The specific mutations are as follows:
NC_000010.10:g.96521657C>T
NC_000010.11:g.94761900C>T
NG_008384.2:g.4195C>T

In yet another embodiment, the allelic variant of UGT1A1 analysed is rs8175347 and/or rs4148323.

Rs8175347 describes variation of a short (TA)(n) repeat sequence (plus strand, GRCh38) covering the TATA box of the UGT1A1 UDP-glucuronosyltransferase1A1 gene.

Examples of variation of the repeat sequence are (TA)7 and (TA)8. The specific mutations are as follows:
NC_000002.11:g.234668881_234668882TA[5][6][7][8];
NC_000002.12:g.233760235_233760236TA[5][6][7][8];
NG_002601.2:g.175492_175493TA[5][6][7][8]; or
NG_033238.1:g.4963_4964TA[5][6][7][8].

Rs4148323 describes three different genotypes, that is A;A, A;G and G;G (plus strand, GRCh38). The specific mutations are as follows:
C_000002.11:g.234669144G>A;
NC_000002.12:g.233760498G>A;
NG_002601.2:g.175755G>A; or
NG_033238.1:g.5226G>A.

Example 2 also demonstrates rs212090 (ABCC1) and rs1045642 (ABCB1) can be used in combination with CYP2D6, CYP2C19 and UGT1A1 to determine a prognosis of a clinical response to CNS-active medicaments in patients suffering from MDD.

In another embodiment, the one or more allelic variants detected are allelic variants of ABCC1, ABCB1 and UGT1A1, wherein the allelic variant of ABCC1 is rs212090 and/or wherein the allelic variant of ABCB1 is rs1045642 and wherein the allelic variant of UGT1A1 is rs8175347 and/or rs4148323.

In another embodiment, the one or more allelic variants detected are allelic variants of ABCC1, ABCB1 and CYP2D6, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6.

In another embodiment, the one or more allelic variants detected are allelic variants of ABCC1, ABCB1, CYP2C19, wherein the allelic variant of ABCC1 is rs212090 and/or wherein the allelic variant of ABCB1 is rs1045642 and/or wherein the allelic variant of CYP2C19 is one or more of rs4244285, rs4986893 and rs12248560.

In another embodiment, the one or more allelic variants detected are allelic variants of ABCC1, ABCB1, CYP2D6 and UGT1A1, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6, and/or wherein the allelic variant of UGT1A1 is rs8175347 and/or rs4148323.

In another embodiment, the one or more allelic variants detected are allelic variants of ABCC1, ABCB1, CYP2C19, and UGT1A1, wherein the allelic variant of ABCC1 is rs212090 and/or wherein the allelic variant of ABCB1 is rs1045642 and/or wherein the allelic variant of CYP2C19 is one or more of rs4244285, rs4986893 and rs12248560, and/or wherein the allelic variant of UGT1A1 is rs8175347 and/or rs4148323

In another embodiment, the one or more allelic variants detected are allelic variants of ABCC1, ABCB1, CYP2D6 and CYP2C19, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6, and/or wherein the allelic variant of CYP2C19 is one or more of rs4244285, rs4986893 and rs12248560.

In another embodiment, the one or more allelic variants detected are allelic variants of ABCC1, ABCB1, CYP2D6, CYP2C19, and UGT1A1, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6, and/or wherein the allelic variant of CYP2C19 is one or more of rs4244285, rs4986893 and rs12248560 and/or wherein the allelic variant UGT1A1 is rs8175347 and/or rs4148323.

The present inventor has demonstrated that particular genotypes are associated with improved or worsened clinical response to CNS-active medicament.

For example, for CYP2D6, the presence of an A;G or G;G allelic variant of rs3892097 and/or the presence of a C;C allelic variant of rs1065852 and/or the presence of a G;G allelic variant of rs28371725 indicates an improved clinical response to the CNS-active medicament. In contrast, the presence of a A;A allelic variant of rs3892097 and/or the presence of a C;T or T;T allelic variant of rs1065852 and/or the presence of a A;A or A;G allelic variant of rs28371725 indicates a worsened clinical response to the CNS-active medicament.

For CYP2C19, the presence of a G;G allelic variant of rs4244285 and/or the presence of a G;G allelic variant of rs4986893 and/or the presence of a C;T or T;T allelic variant of rs12248560 indicates an improved clinical response to the CNS-active medicament. In contrast, the presence of an A;A or A;G allelic variant of rs4244285 and/or the presence of a A;A or A;G allelic variant of rs4986893 and/or the presence of a C;C allelic variant of rs12248560 indicates a worsened clinical response to the CNS-active medicament.

The presence of a TA(7) or TA(8) homozygous allelic variant of rs8175347 (UGT1A1) and/or TT allelic variant of rs4148323 indicate an improved clinical response to the CNS-active medicament.

In another embodiment, the one or more allelic variants detected are allelic variants of ABCC1, ABCB1 and CYP2D6, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6, and wherein the presence of a AA or AT allelic variant of rs212090 and/or the presence of a TT allelic variant of rs1045642, and/or the presence of an A;G or G;G allelic variant of rs3892097, and/or the presence of a C;C allelic variant of rs1065852, and/or the presence of a G;G allelic variant of rs28371725, and wherein the presence of the allelic variants indicate an improved clinical response to the CNS-active medicament.

In another embodiment, the one or more allelic variants detected are allelic variants of ABCC1, ABCB1, and CYP2C19, wherein the allelic variant of ABCC1 is rs212090 and/or wherein the allelic variant of ABCB1 is rs1045642 and/or wherein the allelic variant of CYP2C19 is one or more of rs4244285, rs4986893 and rs12248560, and wherein the presence of a AA or AT allelic variant of rs212090 and/or the presence of a TT allelic variant of rs1045642, and/or the presence of a G;G allelic variant of rs4244285 and/or the presence of a G;G allelic variant of rs4986893 and/or the presence of a C;T or T;T allelic variant, and wherein the presence of the allelic variants indicate an improved clinical response to the CNS-active medicament.

In another embodiment, the one or more allelic variants detected are allelic variants of ABCC1, ABCB1, CYP2D6 and CYP2C19, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6 and/or wherein the allelic variant of CYP2C19 is one or more of rs4244285, rs4986893 and rs12248560, and wherein the presence of a AA or AT allelic variant of rs212090 and/or the presence of a TT allelic variant of rs1045642, and/or the presence of an A;G or G;G allelic variant of rs3892097, and/or the presence of a C;C allelic variant of rs1065852, and/or the presence of a G;G allelic variant of rs28371725, and/or the presence of a G;G allelic variant of rs4244285 and/or the presence of a G;G allelic variant of rs4986893 and/or the presence of a C;T or T;T allelic variant of rs12248560, and wherein the presence of the allelic variants indicate an improved clinical response to the CNS-active medicament.

In another embodiment, the one or more allelic variants detected are allelic variants of ABCC1, ABCB1, CYP2D6, CYP2C19 and UGT1A1, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6 and/or the allelic variant of CYP2C19 is one or more of rs4244285, rs4986893 and rs12248560 and/or the allelic variant of UGT1A1 is rs8175347 and/or rs4148323, and wherein the presence of a AA or AT allelic variant of rs212090 and/or the presence of a TT allelic variant of rs1045642, and/or the presence of an A;G or G;G allelic variant of rs3892097, and/or the presence of a C;C allelic variant of rs1065852, and/or the presence of a G;G allelic variant of rs28371725 and/or the presence of a TA(7) or TA(8) homozygous allelic variant of rs8175347 and/or TT allelic variant of rs8175347, and wherein the presence of the allelic variants indicate an improved clinical response to the CNS-active medicament.

In another embodiment, the one or more allelic variants detected are allelic variants of ABCC1, ABCB1 and UGT1A1 wherein the allelic variant of ABCC1 is rs212090 and/or wherein the allelic variant of ABCB1 is rs1045642 and/or the allelic variant of UGT1A1 is rs8175347 and/or rs4148323, and wherein the presence of a AA or AT allelic variant of rs212090 and/or the presence of a TT allelic variant of rs1045642, and/or TT allelic variant of rs8175347, and wherein the presence of the allelic variants indicate an improved clinical response to the CNS-active medicament.

In another embodiment, the one or more allelic variants detected are allelic variants of ABCC1, ABCB1 and CYP2D6, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6, and wherein the presence of a TT allelic variant of rs212090 and/or the presence of a CC or CT allelic variant of rs1045642, and/or the presence of a A;A allelic variant of rs3892097 and/or the presence of a C;T or T;T allelic variant of rs1065852 and/or the presence of a A;A or A;G allelic variant of rs28371725, and wherein the presence of the allelic variants indicate a worsened or unchanged clinical response to the CNS-active medicament.

In another embodiment, the one or more allelic variants detected are allelic variants of ABCC1, ABCB1, and CYP2C19, wherein the allelic variant of ABCC1 is rs212090 and/or wherein the allelic variant of ABCB1 and/or wherein the allelic variant of CYP2C19 is one or more of rs4244285, rs4986893 and rs12248560, and wherein the presence of a TT allelic variant of rs212090 and/or the presence of a CC or CT allelic variant of rs1045642, and/or the presence of a A;A allelic variant of rs28371725 and/or the presence of an A;A or A;G allelic variant of rs4244285 and/or the presence of a A;A or A;G allelic variant of rs4986893 and/or the presence of a C;C allelic variant of rs12248560, and wherein the presence of the allelic variants indicate a worsened clinical response to the CNS-active medicament.

In another embodiment, the one or more allelic variants detected are allelic variants of ABCC1, ABCB1, CYP2D6 and CYP2C19, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6 and/or wherein the allelic variant of CYP2C19 is one or more of rs4244285, rs4986893 and rs12248560, and wherein the presence of a TT allelic variant of rs212090 and/or the presence of a CC or CT allelic variant of rs1045642, and/or the presence of a A;A allelic variant of rs3892097 and/or the presence of a C;T or T;T allelic variant of rs1065852 and/or the presence of a A;A or A;G allelic variant of rs28371725 and/or the presence of an A;A or A;G allelic variant of rs4244285 and/or the presence of a A;A or A;G allelic variant of rs4986893 and/or the presence of a C;C allelic variant of rs12248560, and wherein the presence of the allelic variants indicate a worsened clinical response to the CNS-active medicament.

In another embodiment, the one or more allelic variants detected are allelic variants of ABCC1, ABCB1, CYP2D6, CYP2C19 and UGT1A1, wherein the allelic variant of ABCC1 is rs212090 and/or wherein the allelic variant of ABCB1 is rs1045642 and/or wherein the allelic variant of CYP2D6 is one or more of rs3892097, rs1065852, rs28371725 and a deletion or duplication of CYP2D6 and/or the allelic variant of CYP2C19 is one or more of rs4244285, rs4986893 and rs12248560 and/or the allelic variant of UGT1A1 is rs8175347 and/or rs4148323 and wherein the presence of a TT allelic variant of rs212090 and/or the presence of a CC or CT allelic variant of rs1045642, and/or the presence of a A;A allelic variant of rs3892097 and/or the presence of a C;T or T;T allelic variant of rs1065852 and/or the presence of a A;A or A;G allelic variant of rs28371725 and/or the presence of an A;A or A;G allelic variant of rs4244285 and/or the presence of a A;A or A;G allelic variant of rs4986893 and/or the presence of a C;C allelic variant of rs12248560 indicates a worsened clinical response to the CNS-active medicament.

The present inventor has demonstrated in two human clinical trials that patients administered CNS-active medicaments exhibit different clinical responses depending on the presence or absence of the allelic variants described herein. Accordingly, the present invention also provides a method described herein, further comprising the step of administering to a patient suffering from major depressive disorder, cyclothymic disorder, or persistent depressive disorder, a CNS-active medicament.

The present inventor has demonstrated the methods described herein allow the dosage of the CNS-active medicament to be adjusted based on the predicted clinical response of the individual. Alternatively, if an individual is predicted to have and unchanged or worsened clinical response to a specific CNS-medicament, a different medicament may be selected for treating that patient.

The word “administered” or “administer” when used to refer to a medicament, means to dispense or apply the medicament to an individual. There are various routes of administration including but not limited to oral, topical, intravenous, subcutaneous, inhalation, etc. The route of administration may be different between different medicaments and the route chosen should be as recommended by the manufacturer.

The dosage of a medicament is the amount of medicament administered at any one time. The recommended dosage of a CNS-active medicament is the dosage the prescribing physician considers appropriate without recourse to the present invention,

In one embodiment of the invention, the CNS-active medicament administered by the method as hereinbefore described is selected from the group comprising sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline.

Example recommended doses of drugs are given in Table 1, below:

TABLE 1
Recommended doses of drugs.

| | Recommended | |
|---|---|---|
| Drug | Dose | |
| Sertraline | 50-200 | mg |
| escitalopram | 10-20 | mg |
| paroxetine | 20-40 | mg |
| fluoxetine | 20-40 | mg |
| fluvoxamine | 50-200 | mg |
| reboxetine | 4-8 | mg |
| venlafaxine | 75-150 | mg |
| desvenlafaxine | 50-100 | mg |
| duloxetine | 30-60 | mg |
| mirtazapine | 30-45 | mg |
| agomelatine | 25-50 | mg |
| clomipramine | 75-200 | mg |
| notriptyline | 50-150 | mg |
| amitriptyline | 75-200 | mg |

The inventor of the present invention has found that the methods described herein can surprisingly determine a prognosis of a clinical response to a CNS-active medicament in a patient suffering from MDD, cyclothymic disorder or persistent depressive disorder. Specifically, the recommended dose of a medicament may be adjusted based on the patient's prognosis. For example, a patient having one or more allelic variants that indicate an improved clinical response is given a dose of the medicament that is lower than the recommended dose and a patient having one or more allelic variants that indicate a worsened clinical response is given a dose of the medicament that is higher than the recommended dose.

In one embodiment, a patient is administered a decreased dose of the CNS-active medicament relative to the recommended dosage of the CNS-active medicament.

For example, in one embodiment, Sertraline is administered at a dose of less than about 50 me, escitalopram is administered at a dose of less than about 10 mg, paroxetine is administered at a dose of less than about 20 mg, fluoxetine is administered at a dose of less than about 20 mg, fluvoxamine is administered at a dose of less than about 50 mg, reboxetine is administered at a dose of less than about 4 mg, venlafaxine is administered at a dose of less than about 75 mg, desvenlafaxine is administered at a dose of less than about 50 mg, duloxetine is administered at a dose of less than about 30 mg, mirtazapine is administered at a dose of less than about 30 mg, agomelatine is administered at a dose of less than about 25 mg, clomipramine is administered at a dose of less than about 75 mg, notriptyline is administered at a dose of less than about 50 mg, and amitriptyline is administered at a dose of less than about 75 mg.

In one embodiment, a patient with an AA or AT allelic variant of rs212090 is administered a decreased dose of the CNS-active medicament relative to the recommended dosage of the CNS-active medicament.

In another embodiment, patients with a TT allelic variant of rs1045642 is administered with a decreased dose of the CNS-active medicament relative to the recommended dosage of the CNS-active medicament.

In a further embodiment, patients with a TA(7) or TA(8) homozygous allelic variant of rs8175347 and/or TT allelic variants of rs8175347, is administered with decreased dose of the CNS-active medicament relative to the recommended dosage of the CNS-active medicaments. (UGT1A1 improved).

In another embodiment, a patients with a A;G or G;G allelic variant of rs3892097 and/or with a C;C allelic variant of rs1065852 and/or with a G;G allelic variant of rs28371725 is administered a decreased dose of the CNS-active medicament relative to the recommended dosage of the CNS-active medicament.

In a further embodiment, patients with a G;G allelic variant of rs4244285 and/or with a G;G allelic variant of rs4986893 and/or with a C;T or T;T allelic variant of rs12248560 is administered with an decreased dose of the CNS-active medicament relative to the recommended dosage of the CNS-active medicaments.

In one embodiment, a patient is administered an increased dose of the CNS-active medicament relative to the recommended dosage of the CNS-active medicament.

For example, in one embodiment, Sertraline is administered at a dose of more than about 200 mg, escitalopram is administered at a dose of more than about 20 mg, paroxetine is administered at a dose of more than about 40 mg, fluoxetine is administered at a dose of more than about 40 mg, fluvoxamine is administered at a dose of more than about 200 mg. reboxetine is administered at a dose of more than about 8 mg, venlafaxine is administered at a dose of more than about 150 mg, desvenlafaxine is administered at a dose of more than about 100 mg, duloxetine is administered at a dose of more than about 60 mg, mirtazapine is administered at a dose of more than about 45 mg, agomelatine is administered at a dose of more than about 50 mg, clomipramine is administered at a dose of more than about 200 mg, notriptyline is administered at a dose of more than about 50 mg, and amitriptyline is administered at a dose of more than about 200 mg

In an embodiment, patients with an TT allelic variant of rs212090 is administered an increased dose of the CNS-active medicament relative to the recommended dosage of the CNS-active medicament.

In yet another embodiment, patients with a CT or CC allelic variant of rs1045642 is administered with an increased dose of the CNS-active medicament relative to the recommended dosage of the CNS-active medicament.

In an embodiment, patients with an A;A allelic variant of rs3892097 and/or with a C;T or T;T allelic variant of rs1065852 and/or with a A;A or A;G allelic variant of rs28371725 is administered an increased dose of the CNS-active medicament relative to the recommended dosage of the CNS-active medicament.

In a further embodiment, patients with an A;A or A;G allelic variant of rs4244285 and/or with a A;A or A;G allelic variant of rs4986893 and/or with a C;C allelic variant of rs12248560 is administered with an increased dose of the CNS-active medicament relative to the recommended dosage of the CNS-active medicaments.

Example 3 demonstrates prediction of the actual dose needed to remit among 95 MDD patients. This method predicted with 86% sensitivity and 96% specificity when a low dose (lesser than or equal to 50 mg) of desvenlafaxine is required to remit. Furthermore, this method predicted with 92% sensitivity and 86% specificity when a medium dose (greater than 50 mg and lesser than or equal to 150 mg) of desvenlafaxine is required to remit. Lastly, this method predicted with 85% sensitivity and 99% specificity when a high dose (greater than 150 mg) of desvenlafaxine is required to remit. Accordingly, in one embodiment, the method comprises determine the presence or absence of an allelic variant of ABCC1, ABCB1 and UGT1A1, wherein the allelic variant of ABCC1 is rs212090 and/or wherein the allelic variant of ABCB1 is rs1045642 and/or wherein the allelic variant of UGT1A1 is rs8175347 and/or rs4148323, and wherein the presence of a AA or AT allelic variant of rs212090 and/or the presence of a TT allelic variant of rs1045642 and/or the presence of a TA(7) or TA(8) homozygous allelic variant of rs8175347 and/or TT allelic variants of rs8175347 indicate an improved clinical response to the CNS-active medicament.

In one embodiment of the present invention, the CNS-active medicament is a substrate of the ABCC1 protein and/or ABCB1 protein, for example, the CNS-active medicament interacts with ABCC1 and/or ABCB1. An example of such an interaction is transport by ABCC1 and/or by ABCB1 of the medicament across the cell membrane.

In an embodiment of the present invention, the presence or absence of an allelic variant is determined by a genotyping analysis method. A number of different methods are known in the field. The genotyping methods known in the art can be broadly classified into four major categories, hybridisation-based methods, enzyme-based methods and methods based on physical properties of DNA and sequencing. Non-limiting examples of hybridisation-based methods include dynamic allele-specific hybridisation, molecular beacons and SNP microarrays. Non-limiting examples of enzyme-based methods include restriction fragment length polymorphism, PCR-based methods, flap endonuclease, primer extension, 5′-nuclease, oligonucleotide ligation assay, etc. Non-limiting examples of methods based on physical properties of DNA include single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay. Non-limiting examples of DNA sequencing methods include next-generation sequencing, pyrosequencing, chain-termination methods, shotgun sequencing, bridge PCR, ion torrent, SOLiD, Sanger sequencing, sequencing by synthesis, etc. It should be appreciated that any sequencing method capable of distinguishing a SNP is suitable for the present invention.

In one embodiment of the present invention, the method as herein described, the improved clinical response may be a delayed, partial, sub-optimal and no clinical response to the CNS-active medicament. A delayed response to the medicament is where the length of time between administering the medicament to the patient and observing a clinical response is longer than the expected given the known pharmacokinetics of the medicament.

In one embodiment, the improved clinical response of the method as hereinbefore described is selected from the group consisting of increased pharmacologic response to the CNS-active medicament, improved treatment of one or more symptoms associated with major depressive disorder, cyclothymic disorder, or persistent depressive disorder, increased remission, decreased medical absence and decreased intolerance to the CNS-active medicament.

The present the invention also provides a method for treating major depressive disorder, cyclothymic disorder, or persistent depressive disorder in a patient, said method comprising determining the presence or absence of an allelic variant of ABCC1, wherein the presence or absence of a allelic variant indicates an improved or worsened clinical response to a CNS-active medicament, and administering to the patient the CNS-active medicament.

In an embodiment of the method for treating major depressive disorder, cyclothymic disorder, or persistent depressive disorder in a patient, the allelic variant of ABCC1 determined in the method for treating major depressive disorder, cyclothymic disorder, or persistent depressive disorder in a patient as hereinbefore described is rs212090.

In a further embodiment of the method for treating major depressive disorder, cyclothymic disorder, or persistent depressive disorder in a patient, the presence or absence of a A;A or A;T allelic variant of rs212090 indicates an improved clinical response to the CNS-active medicament. Accordingly, the patient may be administered with a reduced dose of the CNS-active medicament.

In a further embodiment of the method for treating major depressive disorder, cyclothymic disorder, or persistent depressive disorder in a patient, the presence or absence of a T;T allelic variant of rs212090 indicates a worsened clinical response to the CNS-active medicament. Accordingly, the patient may be administered with an increased dose of the CNS-active medicament.

In a further embodiment of the method for treating major depressive disorder, cyclothymic disorder, or persistent depressive disorder in a patient, further comprises determining the presence or absence of an allelic variant of ABCB1.

In one embodiment, the allelic variant of ABCB1 analysed in the method for treating major depressive disorder, cyclothymic disorder, or persistent depressive disorder in a patient as hereinbefore described is rs1045642.

In a further embodiment of the method for treating major depressive disorder, cyclothymic disorder, or persistent depressive disorder in a patient, the presence or absence of a T;T allelic variant of rs1045642 indicates an improved clinical response to the CNS-active medicament. Accordingly, the patient may be administered with a reduced dose of the CNS-active medicament.

In a further embodiment of the method for treating major depressive disorder, cyclothymic disorder, or persistent depressive disorder in a patient, the presence or absence of a C;C or C;T allelic variant of rs1045642 indicates a worsened clinical response to the CNS-active medicament. Accordingly, the patient may be administered with an increased dose of the CNS-active medicament.

In a further embodiment of the method for treating major depressive disorder, cyclothymic disorder, or persistent depressive disorder in a patient, further comprises determining the presence or absence of an allelic variant of CYP2D6, CYP2C19 and UGT1A1.

In an embodiment, the allelic variant of CYP2D6 analysed in the method for treating major depressive disorder, cyclothymic disorder, or persistent depressive disorder in a patient as hereinbefore described is selected from the group consisting of rs3892097, rs1065852, rs28371725 and a deletion or duplication of P450 2D6.

In an embodiment, the allelic variant of CYP2C19 analysed in the method for treating major depressive disorder, cyclothymic disorder, or persistent depressive disorder in a patient as hereinbefore described is selected from the group consisting of rs4244285, rs4986893 and rs12248560.

In an embodiment, the allelic variant of UGT1A1 analysed in the method for treating major depressive disorder, cyclothymic disorder, or persistent depressive disorder in a patient as hereinbefore described is selected from the group consisting of rs8175347 and rs4148323.

In a further embodiment of the method for treating major depressive disorder, cyclothymic disorder, or persistent depressive disorder in a patient, the presence or absence of a A;G or G;G allelic variant of rs3892097 and/or the presence of a C;C allelic variant of rs1065852 and/or the presence of a G;G allelic variant of rs28371725 indicates an improved clinical response to the CNS-active medicament. Accordingly, the patient may be administered with a reduced dose of the CNS-active medicament.

In a further embodiment of the method for treating major depressive disorder, cyclothymic disorder, or persistent depressive disorder in a patient, the presence or absence of a G;G allelic variant of rs4244285 and/or the presence of a G;G allelic variant of rs4986893 and/or the presence of a C;T or T;T allelic variant of rs12248560 indicates an improved clinical response to the CNS-active medicament. Accordingly, the patient may be administered with a reduced dose of the CNS-active medicament.

In a further embodiment of the method for treating major depressive disorder, cyclothymic disorder, or persistent depressive disorder in a patient, the presence or absence of a TA(7) or TA(8) homozygous allelic variant of rs8175347 and/or TT allelic variants of rs8175347 indicate an improved clinical response to the CNS-active medicament. Accordingly, the patient may be administered with a reduced dose of the CNS-active medicament.

In a further embodiment of the method for treating major depressive disorder, cyclothymic disorder, or persistent depressive disorder in a patient, the presence or absence of a A;A allelic variant of rs3892097 and/or the presence of a C;T or T;T allelic variant of rs1065852 and/or the presence of a A;A or A;G allelic variant of rs28371725 indicates a worsened clinical response to the CNS-active medicament. Accordingly, the patient may be administered with an increased dose of the CNS-active medicament.

In a further embodiment of the method for treating major depressive disorder, cyclothymic disorder, or persistent depressive disorder in a patient, the presence or absence of an A;A or A;G allelic variant of rs4244285 and/or the presence of a A;A or A;G allelic variant of rs4986893 and/or the presence of a C;C allelic variant of rs12248560 indicates a worsened clinical response to the CNS-active medicament. Accordingly, the patient may be administered with an increased dose of the CNS-active medicament.

In a further embodiment of the method for treating major depressive disorder, cyclothymic disorder, or persistent depressive disorder in a patient, an increased dose of the CNS-active medicament relative to the recommended dosage of the CNS-active medicament is administered to the patient having an allelic variant associated with a worsened clinical response.

In a further embodiment of the method for treating major depressive disorder, cyclothymic disorder, or persistent depressive disorder in a patient, a reduced dose of the CNS-active medicament relative to the recommended dosage of the CNS-active medicament is administered to the patient having an allelic variant associated with an improved clinical response.

In a further embodiment of the method for treating major depressive disorder, cyclothymic disorder, or persistent depressive disorder in a patient, the CNS-active medicament is selected from the group comprising sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline.

In a further embodiment of the method for treating major depressive disorder, cyclothymic disorder, or persistent depressive disorder in a patient, the CNS-active medicament is desvenlafaxine.

In a further embodiment of the method for treating major depressive disorder, cyclothymic disorder, or persistent depressive disorder in a patient, the CNS-active medicament is a substrate of the ABCC1 protein and/or ABCB1 protein.

In a further embodiment of the method for treating major depressive disorder, cyclothymic disorder, or persistent depressive disorder in a patient, the presence or absence of an allelic variant is determined by a genotyping analysis comprising the use of mass-spectrometric analysis, microarray analysis, sequencing analysis, polymerase chain reaction and/or polymorphism specific primers.

In a further embodiment of the method for treating major depressive disorder, cyclothymic disorder, or persistent depressive disorder in a patient, the improved clinical response is selected from the group consisting of a delayed, partial, sub-optimal and no clinical response to the CNS-active medicament.

In a further embodiment of the method for treating major depressive disorder, cyclothymic disorder, or persistent depressive disorder in a patient, the improved clinical response is selected from the group consisting of increased pharmacologic response to the CNS-active medicament, improved treatment of one or more symptoms associated with major depressive disorder, cyclothymic disorder, or persistent depressive disorder, increased remission, decreased medical absence and decreased intolerance to the CNS-active medicament.

Another aspect of the present invention provides a kit for determining a prognosis of a clinical response to a CNS-active medicament in a patient suffering from major depressive disorder, cyclothymic disorder, or persistent depressive disorder, said kit comprising a primer or probe for determining the presence or absence of an allelic variant of ABCC1. In one embodiment, the allelic variant of ABCC1 analysed is rs212090.

In a further embodiment, the kit as hereinbefore described further comprises a primer or probe for determining the presence or absence of an allelic variant of ABCB1. In a preferred embodiment, the ABCB1 allelic variant analysed is rs1045642.

In a further embodiment, the kit as hereinbefore described, further comprises a primer or probe for determining the presence or absence of an allelic variant of one or more of CYP2D6, CYP2C19 and UGT1A1. In a preferred embodiment, the CYP2D6 allelic variants analysed is selected from the group consisting of rs3892097, rs1065852, rs28371725 and a deletion or duplication of CYP2D6 and/or the allelic variant of cytochrome CYP2C19 is selected from the group consisting of rs4244285, rs4986893 and rs12248560, and/or the allelic variant of UGT1A1 is rs8175347 or rs4148323.

An aspect of the present invention is a prognostic report generated according to the method for determining a prognosis of a clinical response as hereinbefore described.

Without wishing to be bound by theory, a prognostic report may be generated by assigning weighted values to each of the genotypes described herein and a cumulative score may be provided together with a reference table for determining whether a patient is likely to have a worsened or improved clinical response to the CNS-active medicament. Weighted values may be assigned manually or they may be calculated using a mathematical model, which is trained to classify a clinical score such as remission rate or average days of medical absence, with genotypes and medicament dosage. Non-limiting examples of such mathematical models include principal component analysis, regression modelling, self-organising maps, etc. In one embodiment, the model for generating the report will take ABCC1 genotype into account with one or more of genotypes of ABCB1, CYP2D6, CYP2C19 and UGT1A1. In one embodiment, the relative weight value given to each of the genotype will depend on the original model training data set. In a further embodiment, a report may be generated using more than one model depending on other influencing factors such as ethnicity, age, familial history, etc. The prognosis report may be generally manually by reference to the weighted values or it may be generated automatically by a computer, which performs the calculation.

The present invention also provides a method for determining a therapeutically effective dose of a CNS-active medicament in a patient suffering major depressive disorder, cyclothymic disorder, or persistent depressive disorder, said method comprising determining the presence or absence of an allelic variant of ABCC1, wherein the presence or absence of a allelic variant indicates an improved or worsened clinical response to a CNS-active medicament, and administering to the patient the CNS-active medicament.

In an embodiment, the allelic variant of ABCC1 analysed in the method for determining a therapeutically effective dose of a CNS-active medicament in a patient suffering major depressive disorder, cyclothymic disorder, or persistent depressive disorder as hereinbefore described is rs212090.

In a further embodiment of the method for determining a therapeutically effective dose of a CNS-active medicament in a patient suffering major depressive disorder, cyclothymic disorder, or persistent depressive disorder, the presence or absence of a A;A or A;T allelic variant of rs212090 indicates an improved clinical response to the CNS-active medicament. Accordingly, the patient may be administered with a reduced dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of a T;T allelic variant of rs212090 indicates a worsened clinical response to the CNS-active medicament. Accordingly, the patient may be administered with an increased dose of the CNS-active medicament.

In a further embodiment of the method for determining a therapeutically effective dose of a CNS-active medicament in a patient suffering major depressive disorder, cyclothymic disorder, or persistent depressive disorder, further comprises determining the presence or absence of an allelic variant of ABCB1.

In an embodiment, the allelic variant of ABCB1 analysed in the method for determining a therapeutically effective dose of a CNS-active medicament in a patient suffering major depressive disorder, cyclothymic disorder, or persistent depressive disorder as hereinbefore described is rs1045642.

In a further embodiment of the method for determining a therapeutically effective dose of a CNS-active medicament in a patient suffering major depressive disorder, cyclothymic disorder, or persistent depressive disorder, the presence or absence of a T;T allelic variant of rs1045642 indicates an improved clinical response to the CNS-active medicament. Accordingly, the patient may be administered with a reduced dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of a C;C or C;T allelic variant of rs1045642 indicates a worsened clinical response to the CNS-active medicament. Accordingly, the patient may be administered with an increased dose of the CNS-active medicament.

In a further embodiment of the method for determining a therapeutically effective dose of a CNS-active medicament in a patient suffering major depressive disorder, cyclothymic disorder, or persistent depressive disorder, further comprises determining the presence or absence of an allelic variant of CYP2D6, CYP2C19 and UGT1A1.

In an embodiment, the allelic variant of CYP2D6 analysed in the method for determining a therapeutically effective dose of a CNS-active medicament in a patient suffering major depressive disorder, cyclothymic disorder, or persistent depressive disorder as hereinbefore described is selected from the group consisting of rs3892097, rs1065852, rs28371725 and a deletion or duplication of P450 2D6.

In an embodiment, the allelic variant of CYP2C19 analysed in the method for determining a therapeutically effective dose of a CNS-active medicament in a patient suffering major depressive disorder, cyclothymic disorder, or persistent depressive disorder as hereinbefore described is selected from the group consisting of rs4244285, rs4986893 and rs12248560.

In an embodiment, the allelic variant of UGT1A1 analysed in the method for determining a therapeutically effective dose of a CNS-active medicament in a patient suffering major depressive disorder, cyclothymic disorder, or persistent depressive disorder as hereinbefore described is selected from the group consisting of rs8175347 and rs4148323.

In a further embodiment of the method for determining a therapeutically effective dose of a CNS-active medicament in a patient suffering major depressive disorder, cyclothymic disorder, or persistent depressive disorder, the presence or absence of a A;G or G;G allelic variant of rs3892097 and/or the presence of a C;C allelic variant of rs1065852 and/or the presence of a G;G allelic variant of rs28371725 indicates an improved clinical response to the CNS-active medicament. Accordingly, the patient may be administered with a reduced dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of a G;G allelic variant of rs4244285 and/or the presence of a G;G allelic variant of rs4986893 and/or the presence of a C;T or T;T allelic variant of rs12248560 indicates an improved clinical response to the CNS-active medicament. Accordingly, the patient may be administered with a reduced dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of a TA(7) or TA(8) homozygous allelic variant of rs8175347 and/or TT allelic variants of rs8175347 indicate an improved clinical response to the CNS-active medicament. Accordingly, the patient may be administered with a reduced dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of a A;A allelic variant of rs3892097 and/or the presence of a C;T or T;T allelic variant of rs1065852 and/or the presence of a A;A or A;G allelic variant of rs28371725 indicates a worsened clinical response to the CNS-active medicament. Accordingly, the patient may be administered with an increased dose of the CNS-active medicament.

In a further embodiment of the method for determining a therapeutically effective dose of a CNS-active medicament in a patient suffering major depressive disorder, cyclothymic disorder, or persistent depressive disorder, the presence or absence of an A;A or A;G allelic variant of rs4244285 and/or the presence of a A;A or A;G allelic variant of rs4986893 and/or the presence of a C;C allelic variant of rs12248560 indicates a worsened clinical response to the CNS-active medicament. Accordingly, the patient may be administered with an increased dose of the CNS-active medicament.

In a further embodiment of the method for determining a therapeutically effective dose of a CNS-active medicament in a patient suffering major depressive disorder, cyclothymic disorder, or persistent depressive disorder, an increased dose of the CNS-active medicament relative to the recommended dosage of the CNS-active medicament is administered to the patient having an allelic variant associated with a worsened clinical response.

In a further embodiment of the method for determining a therapeutically effective dose of a CNS-active medicament in a patient suffering major depressive disorder, cyclothymic disorder, or persistent depressive disorder, a reduced dose of the CNS-active medicament relative to the recommended dosage of the CNS-active medicament is administered to the patient having an allelic variant associated with an improved clinical response.

In a further embodiment of the method for determining a therapeutically effective dose of a CNS-active medicament in a patient suffering major depressive disorder, cyclothymic disorder, or persistent depressive disorder, the CNS-active medicament is selected from the group comprising sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline.

In a further embodiment of the method for determining a therapeutically effective dose of a CNS-active medicament in a patient suffering major depressive disorder, cyclothymic disorder, or persistent depressive disorder, the CNS-active medicament is desvenlafaxine.

In a further embodiment of the method for determining a therapeutically effective dose of a CNS-active medicament in a patient suffering major depressive disorder, cyclothymic disorder, or persistent depressive disorder, the CNS-active medicament is a substrate of the ABCC1 protein and/or ABCB1 protein.

In a further embodiment of the method for determining a therapeutically effective dose of a CNS-active medicament in a patient suffering major depressive disorder, cyclothymic disorder, or persistent depressive disorder, the presence or absence of an allelic variant is determined by a genotyping analysis comprising the use of mass-spectrometric analysis, microarray analysis, sequencing analysis, polymerase chain reaction and/or polymorphism specific primers.

In a further embodiment of the method for determining a therapeutically effective dose of a CNS-active medicament in a patient suffering major depressive disorder, cyclothymic disorder, or persistent depressive disorder, the improved clinical response is selected from the group consisting of a delayed, partial, sub-optimal and no clinical response to the CNS-active medicament.

In a further embodiment of the method for determining a therapeutically effective dose of a CNS-active medicament in a patient suffering major depressive disorder, cyclothymic disorder, or persistent depressive disorder, the improved clinical response is selected from the group consisting of increased pharmacologic response to the CNS-active medicament, improved treatment of one or more symptoms associated with major depressive disorder, cyclothymic disorder, or persistent depressive disorder, increased remission, decreased medical absence and decreased intolerance to the CNS-active medicament.

Another aspect of the present invention provides a kit for determining a prognosis of a clinical response to a CNS-active medicament in a patient suffering from major depressive disorder, cyclothymic disorder, or persistent depressive disorder, said kit comprising a primer or probe for determining the presence or absence of an allelic variant of ABCC1. In a preferred embodiment, the allelic variant of ABCC1 analysed is rs212090.

In a further embodiment, the kit further comprises a primer or probe for determining the presence or absence of an allelic variant of ABCB1. In a preferred embodiment, the ABCB1 allelic variant analysed is rs1045642.

In a further embodiment, the kit further comprises a primer or probe for determining the presence or absence of an allelic variant of one or more of CYP2D6, CYP2C19 and UGT1A1. In a preferred embodiment, the CYP2D6 allelic variants analysed is selected from the group consisting of rs3892097, rs1065852, rs28371725 and a deletion or duplication of CYP2D6 and/or the allelic variant of cytochrome CYP2C19 is selected from the group consisting of rs4244285, rs4986893 and rs12248560, and/or the allelic variant of UGT1A1 is rs8175347 or rs4148323.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

The present invention will now be more fully described by reference to the following non-limiting Examples.

### EXAMPLES

### Example 1: Materials and Methods

### Nucleic Acid Extraction

DNA was extracted from patient self-administered buccal brush samples using QIAamp DNA Mini Kit (QIAGEN Inc.) in accordance with the manufacturer's protocol.

### Genotyping

Genotype of candidate SNPs was determined by the polymerase chain reaction (PCR) followed by single primer extension and analysis on a Sequenom® Matrix-assisted laser desorption/ionization time-of-flight mass spectrometry (MALDI-TOF) 384 well genetic analysis system. For the large duplications and deletions standard long-range polymerase chain reaction (PCR) was used.

### Example 2: Improved Clinical Responses to Central Nervous System (CNS)-Active Medicaments in Patients Suffering Major Depressive Disorder Via a Pharmacogenetic Analysis

A double blinded randomized comparator study was to determine the clinically utility of a pharmacogenetic analysis based on determining the presence or absence of allelic variants of ABCC1, ABCB1, CYP2D6, CYP2C19 and/or UGT1A1 in the prognosis of clinical responses to CNS active medicaments and choosing doses of CNS-active medicaments.

The allelic variants examined in this trial were:
For ABCB1—rs1045642;
For ABCC1—rs212090;
For CYP2C19—rs4244285, rs4986893 and rs12248560;
For CYP2D6—rs3892097, rs1065852, rs28371725 and deletion or duplication of CYP2D6;
For UGT1A1—rs8175347 and rs4148323.

This study was performed with a randomized double blind design. Candidate gene association studies have the major drawback of not being able to control for unknown confounders. An advantage of randomization (in large enough samples) is the balancing out between groups of covariates, helping reduce the risk of confounding factors distorting findings.

Subjects received clinical care by their treating psychiatrist during the 12 week study period. Subjects with a principal DSM-5 diagnosis of major depressive disorder (MDD) by semi-structured psychiatrist interview were eligible. Subjects with other active psychiatric diagnoses were excluded, such as those suffering with adjustment disorders, psychosis, bipolar disorder, substance use disorders, and those with a principal diagnosis of a personality disorder. Pregnant or breastfeeding subjects were excluded. Subjects with hepatic or renal impairments were excluded as such could influence appropriate dosing. Subjects co-prescribed known CYP2D6, CYP2C19, or ABCB1 inducers/inhibitors; subjects regularly drinking grapefruit juice; and smokers were also excluded. Age, sex, duration of depressive episode, and number of depressive episodes were recorded as well as baseline 17-item HDRS score. Only subjects with HDRS scores over 18 were included on the basis that moderate to severe depression has robust evidence of antidepressant efficacy. Caucasian subjects were recruited to avoid ethnicity becoming a potential confounder.

All subjects had a buccal brush sample obtained, but via computerized randomization only half had this information analysed and a report sent to their prescriber. Genetic and non-genetic may influence optimal dosing, thus randomization is used to help balance out potential confounding factors between groups. As all subjects provide buccal brush samples, patients were blind to which study group they were in. Antidepressant remission rates were assessed with baseline and 4 weekly HDRS over 12 weeks by an independent rater blinded to which group the patient belonged—genetically guided (n=74) or unguided (n=74). Thus, the study was double blinded—helping prevent patient or outcome assessor biases influencing outcome ratings. The prescriber did not inform the patient if a DNA report was being used in their prescribing, and could indicate (by confidential feedback form) if they elected to use the report information to guide dose and if so doing led them to dose medication differently from usual practice. The antidepressant prescribed was left to the judgement of prescriber and patient—based upon preferred side effect profile and avoidance of agents that had previously proven ineffective or intolerable. This helped ensure patient care was not disadvantaged. A pharmacogenetic interpretive report was prepared following determining each relevant genotype or genotypes to provide a prognostic score, and provides an indication if the patient's genotype suggested mid-range, high-range, or low-range doses were needed. Intolerability events where the patient needed to reduce the dose or stop their antidepressant were recorded for analysis. Finally, the number of sick days taken off from work or studies due to depression was recorded. Ethics approval was obtained from a National Health and Medical Research Council (Australia) approved ethics committee (ACTRN12613001135707). SPSS statistical software (version 22.0) was used in the analysis of the data, results expressed as risk ratios (RR) with 95% confidence interval (CI) and NNG (number need to genotype) values to readily communicate significance and magnitude of any findings. NNG is the number of people that needed the test for an additional patient to remit from MDD. Student's t-test was used to compare differences in averages between groups.

A total of 174 patients were screened for eligibility. A total of 22 subjects were excluded: 12 had a principal diagnosis other than MDD and 10 had a baseline HDRS score <18. The remaining 152 subjects were randomised to either genetically guided or unguided prescribing, but 4 subjects failed to attend for research follow-up. Thus, 148 subjects completed the study, unguided group (n=74) and genetically guided group (n=74) had data analysed for differential antidepressant efficacy and tolerability. Baseline and other characteristics of the sample are displayed in Table 2. Importantly, there were no significant differences in baseline HDRS score to confound findings. There were no significant differences in the antidepressants used between the two groups (Table 3).

TABLE 2
Sample characteristics.

| | Genetically | Genetically | |
|---|---|---|---|
| | guided | unguided | |
| | dosing | dosing | p-value |
| Baseline HDRS | 24.81 | 24.66 | NS |
| Average duration of MDD | 8.51 | 8.59 s | NS |
| (months) | | | |
| Average Number of MDD | 2.22 | 2.18 | NS |
| Episodes | | | |
| Proportion Male (%) | 42% | 39% | NS |
| Average Age (years) | 44.2 | 44.3 | NS |
| Proportion Employed (%) | 91% | 89% | NS |

Clinical and socio-demographic characteristics stratified by genetically guided (patients for which a prognosis was determined on the basis of the presence or absence of the allelic variants described herein) versus unguided prescribing. No significant difference in the above listed characteristics were identified.
NS = Not Significant at p = 0.05 level.
HDRS = 17-item Hamilton Depression Rating Scale.
MDD = Major Depressive Disorder.

TABLE 3
Medication type by group.

| | Genetically | Genetically | |
|---|---|---|---|
| | guided | unguided | |
| Antidepressant | dosing | dosing | p-value |
| Sertraline | 13 | 12 | NS |
| Escitalopram | 8 | 7 | NS |
| Paroxetine | 5 | 7 | NS |
| Fluoxetine | 6 | 7 | NS |
| Fluvoxamine | 2 | 3 | NS |
| Reboxetine | 3 | 5 | NS |
| Venlafaxine | 7 | 7 | NS |
| Desvenlafaxine | 5 | 4 | NS |
| Duloxetine | 5 | 6 | NS |
| Mirtazapine | 5 | 3 | NS |
| Agomelatine | 10 | 8 | NS |
| Clomipramine | 3 | 3 | NS |
| Nortriptyline | 1 | 1 | NS |
| Amitriptyline | 1 | 1 | NS |

Choice of medication was at the discretion of the prescriber and subject. There were no significant differences in the frequencies of different antidepressants used between the genetically guided and unguided groups.
NS = Not Significant at the p = 0.05 level).

A pharmacogenetic interpretive report was reviewed by the treating prescriber in 100% (74/74) of instances. This led to medication dosing different to usual practice by the prescriber 65% (48/74) of the time. Table 4 displays the key study findings.

The “genetically guided” group in which a prognosis was determined on the basis of the presence or absence of the allelic variants described herein was 2.52 times more likely to remit from MDD (95% Cl=1.71-3.73, z=4.66, p<0.0001) than the unguided group, with NNG for remission from MDD=3 (95% CI 1.7-3.5). The unguided group were 1.13 times more likely to have medication tolerability problems (95% Cl=1.01-1.25, z=2.208, p=0.0272) requiring either dose reduction or cessation. The genetically guided group had significantly less risk of taking sick leave (4% versus 15%, p=0.0272) and significantly less duration of sick leave when such was needed (4.3 days versus 7.7 days, p=0.014).

TABLE 4
Differential antidepressant efficacy and tolerability by group.

| | | | Proportion | |
|---|---|---|---|---|
| | Remission | Intolerability | Taking Sick | Average Sick |
| | Rate | Rate | Leave | Days |
| Genetically | 72% | 4% | 4% | 4.3 |
| guided | RR = 2.52 | | | (p = 0.014) |
| dosing | (95% CI = | | | |
| | 1.71-3.73, | | | |
| | z = 4.660, | | | |
| | p < 0.0001) | | | |
| | NNG = 3 | | | |
| | (95% CI = | | | |
| | 1.7-3.5) | | | |
| Unguided | 28% | 15% | 15% | 7.7 |
| dosing | | RR = 1.13 | RR = 1.13 | |
| | | (95% CI = | (95% CI = | |
| | | 1.01-1.25, | 1.01-1.25, | |
| | | z = 2.208, | z = 2.208, | |
| | | p = 0.0272) | p = 0.0272) | |
| | | NNG = 10 | | |
| | | (95% CI = | | |
| | | 5.0-64.8) | | |

The genetically guided group in which a prognosis was determined on the basis of the presence or absence of the allelic variants described herein had significantly greater remission rates, better tolerability, and fewer sick days from work. Those subjects treated with genetically guided dosing had a 2.52-fold greater chance of remission form MDD (HDRS≤7) and a 1.13-fold reduced risk of medication intolerability (dose reduction or cessation needed).
NNG = Number Needed to Genotype.

This data demonstrates that the presence or absence of allelic variants of ABCC1, ABCB1, CYP2D6, CYP2C19 and UGT1A1 can be used to determine a prognosis of a clinical response to a CNS-active medicament.

The clinical responses examined included remission, pharmacologic response to the CNS-active medicament, intolerance to the CNS-active medicaments, proportion taking sick leave (medical absence) and average sick days. It is clear from the data in Table 4, the remission rate in the genetically guided (prognosis determined) group is nearly double that found in the current (unguided) dosing practice.

Because remission is a pathway to recovery from MDD, the markedly improved remission rates (72% versus 28%) in the genetically guided group has significant clinical implications. A second clinical response measured was tolerance to the administered drug, or intolerability rate. Genetically guided dosing (based on the prognosis determined) provided a significantly reduced rate of intolerance to the administered drug (4% versus 15% of the unguided dosing). A third clinical response measured was the rate of sick leave and the duration of sick leave. The data also demonstrates that genetically guided dosing provided a reduced percentage of individuals taking sick leave (or medical absence), and a reduction in the average duration of any sick leave taken.

The clinical trial demonstrated the clinical utility of determining a prognosis of a clinical response to a CNS-active medicament in patients suffering from major depressive disorder, based on the presence or absence of allelic variants of ABCC1, ABCB1, CYP2D6, CYP2C19 and UGT1A1, including to guide the dosage of CNS-active medicaments. Patients whose CNS-active medicament dose was adjusted based on the result of their pharmacogenetic test had higher remission rates, fewer days off work due to depression, shorter average length of medical leave, and better antidepressant tolerability (fewer instances of side effects requiring dose reduction or medication cessation).

In particular, the data demonstrates that the presence or absence of allelic variants of ABCC1, ABCB1, CYP2D6, CYP2C19 and UGT1A1 can be used to determine a prognosis of a clinical response to Sertraline, Escitalopram, Paroxetine, Fluoxetine, Fluvoxamine, Reboxetine, Venlafaxine and Desvenlafaxine in patients suffering from major depressive disorder.

This study is also the first statistically significant positive double blind randomized genetically guided versus unguided prospective comparator trial demonstrating that a pharmacogenetic interpretive report (determining a prognosis) improves antidepressant efficacy by guiding the administered dose.

In particular, the data demonstrates that the presence or absence of allelic variants of ABCC1, ABCB1, CYP2D6, CYP2C19 and UGT1A1 can be used to determine a prognosis of a clinical response to different doses of Sertraline, Escitalopram, Paroxetine, Fluoxetine, Fluvoxamine, Reboxetine, Venlafaxine and Desvenlafaxine in patients suffering from major depressive disorder.

### Example 3: Improved Clinical Responses, Including Remission, to Desvenlafaxine in Patients Suffering Major Depressive Disorder Via a Pharmacogenetic Analysis and Predicting Desvenlafaxine Dose Needed to Achieve Remission in Major Depressive Disorder

A 12-week double-blind randomized clinical trial was performed to examine the clinical validity of a five gene, combinatorial, pharmacogenetic-based antidepressant dosing support tool for desvenlafaxine dosing.

Participants were self-identified Caucasian outpatients aged 18 years and older with a principal DSM-5 diagnosis of MDD (semi-structured psychiatrist assessment) and a 17-item Hamilton Depression Rating Scale (HDRS) score greater than or equal to 18. Participants with other active psychiatric diagnoses were excluded, specifically those suffering with adjustment disorder with depressed mood, persistent depressive disorder, and subjects with a principal clinical diagnosis of a personality disorder. Additional exclusion criteria included pregnancy or breastfeeding, hepatic or renal impairments, co-prescription of known UGT1A1 or ABCB1 inducers/inhibitors (e.g. St John's Wort, valproic acid), regular grapefruit juice consumption, and current smoking which may influence appropriate dosing. A total of 131 individuals were screened for eligibility criteria. Seven subjects did not meet inclusion/exclusion criteria and an additional five failed to return for inclusion in the study, resulting in a final study sample of 119 participants.

### Study Procedures

All participants received desvenlafaxine in an open-label manner during the 10-week study period. At baseline, age, sex, duration of the current depressive episode, and number of depressive episodes was recorded. Desvenlafaxine dose was increased, decreased, or left unchanged every two-weeks (from baseline) based on subjectively reported tolerability and clinical assessment of symptom improvement. Symptom severity was assessed at baseline and every two-weeks thereafter during the study period with the HDRS. Remission was defined as an HDRS score of 7 or less by week 10 of the study. Physicians and the symptom rater were blinded to genotypes. All participants gave written informed consent and procedures were in accordance with the Declaration of Helsinki and were approved by an ethics committee at Deakin University, Australia.

The allelic variants examined in this trial were:
For ABCB1—rs1045642;
For ABCC1—rs212090;
For UGT1A1—rs8175347 and rs4148323.

The allelic variants of the above genes were determined for each patient (the ‘pharmacogenetic analysis’, and used to predict each participant's optimal desvenlafaxine dose range as low (50 mg), medium (>50 mg and <150 mg), or high (150 mg). For this study, CYP2C19 and CYP2D6 allelic variants were determined but were not used to predict desvenlafaxine dose.

DNA was extracted from participant self-administered buccal brush samples using QIAamp DNA Mini Kit (QIAGEN Inc., Chadstone, Victoria, Australia). Genotyping was determined by polymerase chain reaction followed by single primer extension and analysis on a Sequenom® Matrix-assisted laser desorption/ionization time-of-flight mass spectrometry 384 well genetic analysis system by Healthscope Molecular (Clayton, Victoria, Australia).

### Analysis

Among remitters, the performance of the pharmacogenetic analysis was estimated by comparing the predicted desvenlafaxine dosing range derived from the pharmacogenetic analysis with the actual desvenlafaxine dose required to achieve symptom remission. The non-parametric Kendall's tau b (T_{b}) correlation coefficient was used to estimate concordance between actual and predicted dose. Sensitivity, specificity, false positive and false negative rates, as well as accuracy of the pharmacogenetic analysis predicted dose range relative to the actual dose range were also calculated. Additionally, individual genes/variants comprising the pharmacogenetic analysis were compared to the actual dose required for remission to determine if any one gene/variant performed better than the combinatorial pharmacogenetic analysis. Among non-remitters who showed a 50% reduction in HDRS score from baseline, exploratory analyses were conducted using the same analytical methods used in the remitted sample.

### Results

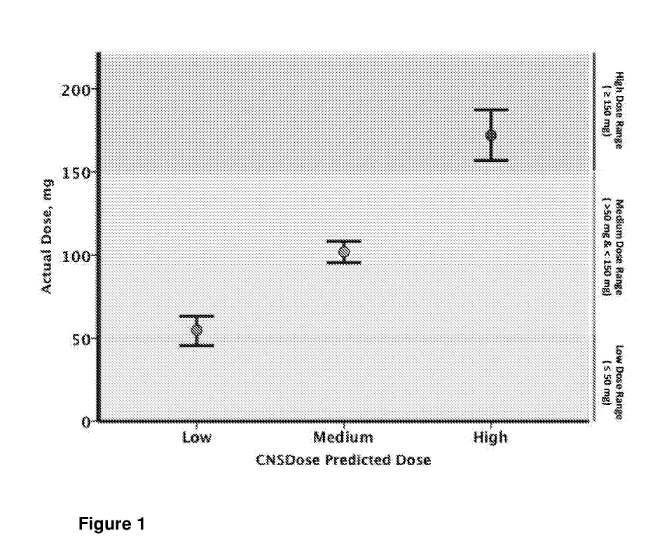

After 10 weeks of desvenlafaxine treatment 80% (n=95) of participants achieved symptom remission (Table 5). Of the 95 participants who achieved symptom remission, 22 (23%) received a low dose; 53 (56%) received a medium dose, and 20 (21%) received a high dose. The pharmacogenetic analysis predicted 22 (23%) required a low dose, 55 (58%) required a medium dose, and 18 (19%) required a high dose to remit. Comparison of the actual and the pharmacogenetic analysis predicted doses required for remission revealed strong concordance (T_{b}=0.84, p=0.0001) (FIG. 1).

In addition, the pharmacogenetic analysis predicted dose showed high sensitivity (85%-92%), specificity (86%-92%), and accuracy (89%-96%) relative to the actual dose required for symptom remission (Table 6). Examination of the individual genes/variants included in the pharmacogenetic analysis showed concordance between the actual dose and predicted dose for ABCB1 (T_{b}=0.54, p=0.0001) and ABCC1 (T_{b}=0.48, p=0.0001) but weak concordance for UGT1A1 (T_{b}=0.14, p=0.149). Sensitivity and specificity of each individual gene/variant was more variable and accuracy estimates lower than observed for the combinatorial pharmacogenetic analysis (Table 7).

TABLE 5
Participant characteristics by remission status.

| | | | Non- |
|---|---|---|---|
| | Full Sample | Remitters | Remitters |
| Characteristic | (n = 119) | (n = 95) | (n = 24) |
| Age, mean (sd) | 49 (13) | 48 (13) | 50 (13) |
| Sex, % (n) females* | 56 (67) | 61 (58) | 38 (9) |
| MDD Episode duration, | 10 (5) | 10 (5) | 10 (3) |
| mean (sd) months | | | |
| MDD episodes, mean (sd) | 2 (1) | 2 (1) | 2 (1) |
| Baseline HDRS-17 score, | 24 (4) | 24 (4) | 24 (4) |
| mean (sd) | | | |
| Final desvenlafaxine dose, | 108 (46) | 104 (49) | 122 (45) |
| mean (sd) mg | | | |

*remitters vs. non-remitters (chi-square = 4.32, df = 1, p = 0.038)

TABLE 6
Performance of pharmacogenetic analysis in predicting required
desvenlafaxine dose needed to remit among 95 MDD remitters.

| Pharmaco- | | | |
|---|---|---|---|
| genetically | | | |
| Predicted | Actual Dose to Remit | | |
| Dose to Remit | Low | Medium | High |
| Low | n = 19 | n = 3 | n = 0 |
| Medium | n = 3 | n = 49 | n = 3 |
| High | n = 0 | n = 1 | n = 17 |
| Performance, | | | |
| estimate | | | |
| (95% CI) | | | |
| Sensitivity | 86% (65%-97%) | 92% (82%-98%) | 85% (62%-97%) |
| (true positive | | | |
| rate) | | | |
| Specificity | 96% (88%-99%) | 86% (71%-95%) | 99% (93%-100%) |
| (true negative | | | |
| rate) | | | |
| False Positive | 4% (1%-12%) | 14% (5%-29%) | 1% (0%-7%) |
| Rate | | | |
| False Negative | 14% (3%-35%) | 8% (2%-18%) | 15% (3%-38%) |
| Rate | | | |
| Accuracy | 94% (89%-99%) | 89% (83%-95%) | 96% (92%-100%) |

TABLE 7
Individual gene* performance in predicting required
desvenlafaxine dose needed to remit among 95 MDD remitters.

| Performance, | | | |
|---|---|---|---|
| value | Actual Dose to Remit | | |
| (95% CI) | Low | Medium | High |
| ABCB1 | | | |
| Sensitivity | 81% (60%-95%) | 66% (52%-78%) | 40% (19%-64%) |
| Specificity | 84% (73%-91%) | 64% (48%-78%) | 91% (82%-96%) |
| Accuracy | 83% (75%-91%) | 65% (55%-75%) | 80% (72%-88%) |
| ABCC1 | | | |
| Sensitivity | 18% (5%-40%) | 57% (42%-70%) | 90% (63%-99%) |
| Specificity | 84% (73%-91%) | 52% (36%-68%) | 85% (75%-92%) |
| Accuracy | 68% (59%-77%) | 55% (45%-65%) | 86% (79%-93%) |
| UGT1A1 | | | |
| Sensitivity | 23% (8%-45%) | 98% (90%-100%) | # |
| Specificity | 95% (87%-98%) | 19% (9%-34%) | # |
| Accuracy | 78% (70%-86%) | 63% (53%-73%) | # |

*Genetic variation in CYP2C19 and CYP2D6 were included in the pharmacogenetic analysis but are not used in predicting the dosing range for desvenlafaxine, and are therefore not shown in the table.
# UGT1A1 ultra-rapid metabolizer phenotype is rare (<1%) in all ethnicities except Africans (prevalence 3.5%) and thus a high dose would not be predicted based on this gene alone.

Among the 24 participants who did not achieve symptom remission by week 10, 42% (n=10) had a greater than 50% reduction in HDRS from baseline. Among these non-remitted responders, 2 (20%) received a low dose, 6 (60%) received a medium dose, and 2 (20%) received a high dose. The pharmacogenetic analysis predicted 3 (30%) would require a low dose, 5 (50%) a medium dose, and 2 (20%) a high dose. Similar to the remitter analysis, comparison of the actual and the pharmacogenetically predicted doses required for response revealed strong concordance (T_{b}=0.87, p=0.004). Performance estimates (e.g. sensitivity, specificity, and accuracy) were not calculated within the non-remitted responder sample given the extremely small sample size.

These results suggest the combinatorial pharmacogenetic analysis have clinical utility in guiding desvenlafaxine dosing in a subset of individuals with moderate to severe depressive symptoms. There is a high degree of concordance (Tb=0.84) between the actual and pharmacogenetic analysis predicted dose required for symptom remission with accuracy ranging from 89%-96%.

This data demonstrates statistically significant concordance between the actual dose and predicted dose of desvenlafaxine for the allelic variants of ABCB1 and ABCC1. Accordingly, this data demonstrates that the presence or absence of allelic variants of ABCC1 and/or ABCB1, can be used to determine a prognosis of a clinical response to a CNS-active medicament, in patients suffering from major depressive disorder.

This data also demonstrates that the presence or absence of allelic variants of ABCC1, and ABCB1, combined with UGT1A1, can be used to improve sensitivity and specificity when determining a prognosis of a clinical response to a CNS-active medicament (Table 6). This result is surprising since UGT1A1 alone did not show statistically significant concordance with the actual dose and predicted dose of desvenlafaxine.

The data also demonstrates that the presence or absence of allelic variants of ABCC1, ABCB1 and UGT1A1 can be used to determine a prognosis of a clinical response to different doses of a CNS-active medicament in patients suffering from major depressive disorder.

1. A method of determining a prognosis of a clinical response to a central nervous system (CNS)-active medicament in a patient suffering from major depressive disorder, cyclothymic disorder or persistent depressive disorder, said method comprising determining the presence or absence of an allelic variant of ABCC1; wherein the presence or absence the allelic variant indicates an improved or worsened clinical response to the CNS-active medicament.

2. The method of claim 1, wherein the allelic variant of ABCC1 is rs212090.

3. The method of claim 1, wherein the presence of an A;A or A;T allelic variant of rs212090 indicates an improved clinical response to the CNS-active medicament.

4. The method of claim 1, wherein the presence of a T;T allelic variant of rs212090 indicates a worsened clinical response to the CNS-active medicament.

5. The method of claim 1, further comprising determining the presence or absence of an allelic variant of ABCB1.

6. The method of claim 5, wherein the allelic variant of ABCB1 is rs1045642.

7. The method of claim 6, wherein the presence of a T;T allelic variant of rs1045642 indicates an improved clinical response to the CNS-active medicament.

8. The method of claim 6, wherein the presence of a C;C or C;T allelic variant of rs1045642 indicates a worsened clinical response to the CNS-active medicament.

9. The method of any one of the preceding claims, wherein the method further comprises determining the presence or absence of an allelic variant selected from the group consisting of CYP2D6, CYP2C19 and UGT1A1.

10. The method of claim 9, wherein the allelic variant of CYP2D6 is selected from the group consisting of rs3892097, rs1065852, rs28371725 and a deletion or duplication of P450 2D6.

11. The method of claim 9 or claim 10, wherein the allelic variant of CYP2C19 is selected from the group consisting of rs4244285, rs4986893 and rs12248560.

12. The method of claim 9, 10 or 11, wherein the allelic variant of UGT1A1 is selected from the group consisting of rs8175347 and rs4148323.

13. The method of any one of claims 10 to 12, wherein the presence of an A;G or G;G allelic variant of rs3892097 and/or the presence of a C;C allelic variant of rs1065852 and/or the presence of a G;G allelic variant of rs28371725 indicates an improved clinical response to the CNS-active medicament.

14. The method of any one of claims 10 to 12, wherein the presence of a G;G allelic variant of rs4244285 and/or the presence of a G;G allelic variant of rs4986893 and/or the presence of a C;T or T;T allelic variant of rs12248560 indicates an improved clinical response to the CNS-active medicament.

15. The method of claim 12, wherein the presence of a TA(7) or TA(8) homozygous allelic variant of rs8175347 and/or TT allelic variants of rs8175347 indicate an improved clinical response to the CNS-active medicament.

16. The method of any one of claims 10 to 12, wherein the presence of an A;A allelic variant of rs3892097 and/or the presence of a C;T or T;T allelic variant of rs1065852 and/or the presence of an A;A or A;G allelic variant of rs28371725 indicates a worsened clinical response to the CNS-active medicament.

17. The method of any one of claims 10 to 12, wherein the presence of an A;A or A;G allelic variant of rs4244285 and/or the presence of an A;A or A;G allelic variant of rs4986893 and/or the presence of a C;C allelic variant of rs12248560 indicates a worsened clinical response to the CNS-active medicament.

18. The method of any one of the preceding claims, further comprising the step of administering to a patient having an allelic variant associated with an improved clinical response a decreased dose of the CNS-active medicament relative to the recommended dosage of the CNS-active medicament.

19. The method of any one of the preceding claims, further comprising the step of administering to a patient having an allelic variant associated with a worsened clinical response an increased dose of the CNS-active medicament relative to the recommended dosage of the CNS-active medicament.

20. The method of any one of the preceding claims, wherein the CNS-active medicament is selected from the group comprising sertraline, escitalopram, paroxetine, fluoxetine, fluvoxamine, reboxetine, venlafaxine, desvenlafaxine, duloxetine, mirtazapine, agomelatine, clomipramine, notriptyline and amitriptyline.

21. The method of claim 20, wherein the CNS-active medicament is desvenlafaxine.

22. The method of any one of the preceding claims, wherein the CNS-active medicament is a substrate of the ABCC1 protein and/or ABCB1 protein.

23. The method of any one of the preceding claims, wherein the presence or absence of an allelic variant is determined by a genotyping analysis comprising the use of mass-spectrometric analysis, microarray analysis, sequencing analysis, polymerase chain reaction and/or polymorphism specific primers.

24. The method of any one of the preceding claims, wherein the improved clinical response is selected from the group consisting of a delayed, partial, sub-optimal and no clinical response to the CNS-active medicament.

25. The method of any one of claims 1 to 23, wherein the improved clinical response is selected from the group consisting of increased pharmacologic response to the CNS-active medicament, improved treatment of one or more symptoms associated with major depressive disorder, cyclothymic disorder, or persistent depressive disorder, increased remission, decreased medical absence and decreased intolerance to the CNS-active medicament.

26. A kit for determining a prognosis of a clinical response to a CNS-active medicament in a patient suffering from major depressive disorder, cyclothymic disorder, or persistent depressive disorder, said kit comprising a primer or probe for determining the presence or absence of an allelic variant of ABCC1.

27. The kit according to claim 26, wherein the allelic variant of ABCC1 is rs212090.

28. The kit according to claim 26 or 27, further comprising a primer or probe for determining the presence or absence of an allelic variant of ABCB1.

29. The kit according to claim 28, wherein the allelic variant of ABCB1 is rs1045642.

30. The kit according to any one of claims 26 to 29, further comprising a primer or probe for determining the presence or absence of an allelic variant of one or more of CYP2D6, CYP2C19 and UGT1A1.

31. The kit according to claim 30, wherein the allelic variant of CYP2D6 is selected from the group consisting of rs3892097, rs1065852, rs28371725 and a deletion or duplication of P450 2D6; the allelic variant of CYP2C19 is selected from the group consisting of rs4244285, rs4986893 and rs12248560, and/or the allelic variant of UGT1A1 is rs8175347 or rs4148323.

32. A prognostic report generated according to the method of any one of claims 1 to 17.
